(19) United States
(12) Patent Application Publication
(10) Pub. No.: US 2004/0033552 A1
(43) Pub. Date: Feb. 19, 2004

(54) Method of detecting and reducing boar taint

(76) Inventors: E. James Squires, Guelph (CA); Jakub Babol, Upssala (SE); Kerstin Lundstrom, Upssala (SE)

Correspondence Address: Micheline Gravelle, Bereskin & Parr, 40 King Street West, Box 401, Toronto, M5H 3Y2, CA

(21) Appl. No.: 10/024,628

(22) Filed: Nov. 27, 2001

Related U.S. Application Data
(63) Continuation-in-part of application No. 09/288,037, filed on Apr. 8, 1999, now abandoned.
(60) Provisional application No. 60/081,037, filed on Apr. 8, 1998.

(51) Int. Cl. C12Q 1/48; A01K 67/027

(52) U.S. Cl. 435/15; 800/17

(57) ABSTRACT

A method of preventing or reducing boar taint is described. The method involves increasing the metabolism of skatole in a pig by modulating the activity or expression of the enzymes involved in skatole metabolism.

This application is a continuation-in-part of U.S. patent application Ser. No. 09/288,037, filed on Apr. 8, 1999 which claims the benefit of United States provisional patent application No. 60/081,037, filed on Apr. 8, 1998, now abandoned, both of which are incorporated by reference.

### FIELD OF THE INVENTION

The invention relates to methods for detecting and preventing boar taint.

### BACKGROUND OF THE INVENTION

Male pigs that are raised for meat production are usually castrated shortly after birth to prevent the development of off-odors and off flavors (boar taint) in the carcass. Boar taint is primarily due to high levels of either the 16-androstene steroids (especially 5α-androst-16-en-3-one) or skatole in the fat. Recent results of the EU research program AIR 3-PL94-2482 suggest that skatole contributes more to boar taint than androstenone (Bonneau, M., 1997).

Skatole is produced by bacteria in the hindgut which degrade tryptophan that is available from undigested feed or from the turnover of cells lining the gut of the pig (Jensen and Jensen, 1995). Skatole is absorbed from the gut and metabolised primarily in the liver Jensen and Jensen, 1995). High levels of skatole can accumulate in the fat, particularly in male pig, and the presence of a recessive gene Ska¹, which results in decreased metabolism and clearance of skatole has been proposed (Lundström et al, 1994; Friis, 1995). Skatole metabolism has been studied extensively in ruminants (Smith, et al., 1993), where it can be produced in large amounts by ruminal bacteria and results in toxic effects on the lungs (reviewed in Yost, 1989). The metabolic pathways involving skatole have not been well described in pigs, In particular, the reasons why only some intact male pigs have high concentrations of skatole in the fat are not clear. Environmental and dietary factors are important (Kjeldsen, 1993; Hansen et al., 1995) but do not sufficiently explain the reasons for the variation in fat skatole concentrations in pigs. Claus et al. (1994) proposed high fat skatole concentrations are a result of an increased intestinal skatole production due to the action of androgens and glucocorticoids. Lundström et al. (1994) reported a genetic influence on the concentrations of skatole in the fat, which may be due to the genetic control of the enzymatic clearance of skatole. The liver is the primary site of metabolism of skatole and liver enzymatic activities could be the controlling factor of skatole deposition in the fat. Bæk et al.(1995) described several liver metabolites of skatole found in blood and urine with the major being MII and MIII. MII, which is a sulfate conjugate of 6-hydroxyskatole (pro-MII), was only found in high concentrations in plasma of pigs which were able to rapidly clear skatole from the body, whereas high MIII concentrations were related to slow clearance of skatole Thus the capability of synthesis of MII could be a major step in a rapid metabolic clearance of skatole resulting in low concentrations of skatole in fat and consequently low levels of boar taint.

In view of the foregoing, further work is needed to fully understand the metabolism of skatole in pig liver and to identify the key enzymes involved. Understanding the biochemical events involved in skatole metabolism can lead to novel strategies for treating, reducing or preventing boar taint. In addition, polymorphisms in these candidate genes may be useful as possible markers for low boar taint pigs.

### SUMMARY OF THE INVENTION

Broadly stated, the present invention relates to methods for determining the susceptibility of a pig to boar taint as well as to a method for reducing or preventing boar taint in a male pig.

The metabolism of skatole in pigs involves Phase I oxidation reactions carried our by cytochrome P450, in particular CYP2E1, and Phase II conjugation reactions carried out by glucuronyl transferases, sulfotransferases, in particular thermostable phenol sulfotransferase and glutathione transferases.

With regard to the Phase I reactions, the inventors have shown that increased expression of CYP2E1 in the livers of pigs is correlated with low levels of skatole in the fat. The inventors have also shown that synthesis of metabolite F-1 (indole-3-carbinol) is correlated with low levels of skatole in the fat.

Accordingly, in one aspect, the present invention provides a method for assessing the ability of a pig to metabolise skatole comprising (a) obtaining a sample from the pig and (b) detecting the levels of CYP2E1 in the sample wherein high levels of CYP2E1 indicates that the pig is a good skatole atetaboliser. In another aspect, the present invention provides a method for determining the susceptibility of a male pig to boar taint comprising (a) obtaining a sample from the pig and (b) detecting the levels of CYP2E1 in the sample, wherein high levels of CYP-E1 indicates that the pig has a reduced susceptibility to developing boar taint. In a further aspect, the present invention provides a method for reducing boar taint comprising enhancing the activity of CYP2E1 in a pig. The activity of CYP2E1 can be enhanced by using substances which (a) increase The activity of CYP2E1; or O) induce or increase the expression of the CYP2E1 gene.

With regard to the Phase II reactions, the inventors have shown that the formation and sulfation of the metabolite 6-hydroxy-3-methylindole (pro-MII) is related to low far skatole levels while high rates of glucuronidation were correlated with high skatole levels in fat. The sulfation of the skatole metabolite 5-hydroxyskatole is due to a thermostable form of phenol sulfotransferase.

Accordingly, in another aspect, the present invention provides a method for assessing the ability of a pig to metabolise skatole comprising (a) obtaining a sample from the pig and (b) detecting the levels of thermostable phenol sulfotransferase in the sample wherein high levels of thermostable phenol sulfotransferase indicates that the pig is a good skatole metaboliser. In another aspect, the present invention provides a method for determining the susceptibility of a male pig to boar taint comprising (a) obtaining a sample from the pig and (b) detecting the levels of thermostable phenol sulfotransferase in the sample, wherein high levels of thermostable phenol sulfotransferase indicates that the pig has a reduced susceptibility to developing boar taint. In a further aspect, the present invention provides a method for reducing boar taint comprising enhancing the activity of thermostable phenol sulfotransferase in a pig.

In a further aspect, the present invention provides a method a method for assessing the ability of a pig to metabolise skatole comprising (a) obtaining a sample from the pig and b) detecting the levels of glucuronyl transferase in the sample wherein low levels of glucuronyl transferase indicates that the pig is a good skatole metaboliser. In another aspect, the present invention provides a method for determining the susceptibility of a male pig to boar taint comprising (a) obtaining a sample from the pig and (b) detecting the levels of glucuronyl transferase in the sample, wherein low levels of glucuronyl transferase indicates that the pig has a reduced susceptibility to developing boar taint. In a further aspect, the present invention provides a method for reducing boar taint comprising inhibiting the activity of a glucuronyl transferase in a pig.

The present invention also includes a method of screening for a substance that regulates skatole metabolism in a pig. In one embodiment, the present invention provides a method for screening a substance that activates CYP2E1 activity or induces transcription and/or translation of the gene encoding CYP2E1. In another embodiment, the present invention provides a method for screening for a substance that enhances the activity of thermostable phenol sulfotransferase or enhances the transcription or translation of a gene encoding a thermostable phenol sulfotransferase in a pig. In a further embodiment, the present invention includes a method for screening for a substance that inhibits glucuronidation activity or inhibits the transcription or translation of a gene encoding a glucuronyl transferase in a pig.

The present invention also includes a pharmaceutical composition for use in treating boar taint comprising an effective amount of a substance which regulates skatole metabolism in a pig and/or a pharmaceutical acceptable carrier, diluent or excipient.

The present invention further includes a method for producing pigs that have a lower incidence of boar taint comprising selecting pigs that express high levels of CYP2E1 and/or thermostable phenol sulfotransferase and/or low levels of glucuronyl transferase; and breeding the selected pigs.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 is a HPLC chromatogram of skatole metabolites produced by liver microsomes as detected by UV absorbance, (A) and fluorescence, (B). Peaks UV-2 and F4 were identified as MIII (3hydroxy-3-methyloxindole) and pro-MII (6-hydroxyskatole) respectively; IS, internal standard (indole3-acetonitrile); SK, skatole; UV-1 (3-hydroxy-3-methylindolenine), UV-3 (3-methyloxindole), F-1 (indole-3-carbinol), F-3 was not identified.

FIG. 2 is a graph showing the formation of skatole metabolites in liver microsomes. Metabolite F-1 is not presented because of its much larger peak area than that of the other metabolites Its synthesis pattern over time followed that of the other metabolites. Each data point represents the mean of duplicate assays performed for 2 pigs.

FIG. 3 is a graph showing the effect of SKF 525A on formation of skatole metabolites in liver microsomes. Each data point represents the mean of duplicate assays performed for 3 pigs.

FIG. 4 is a graph showing the effect of metyrapone on formation of skatole metabolites in liver microsomes. Each data point represents the mean of duplicate assays performed for 3 pigs.

FIG. 5 is a graph showing the effect of DAS on formation of skatole metabolites in liver microsomes. Each data point represents the mean of duplicate assays performed for 3 pigs.

FIG. 6 is a graph showing the effect of chlorzoxazone on formation of skatole metabolites in liver microsomes. Each data point represents the mean of duplicate assays performed for 3 pigs.

FIG. 7 is a graph showing the levels of skatole in fat versus the levels of CYP2E1 in liver. The open symbols are data from Yorkshire pigs and the filled symbols are data from the Wild Pig crosses.

FIG. 8 is a graph showing the glucuronidation of liver microsomal metabolites of skatole. Each data point represents the mean of duplicate assays performed for 3 pigs. UDPCA is uridine 5′-diphosphoglucuronyl. **Differs from control, P<0.01; ***differs from control, P<0.001.

FIG. 9 is a graph showing the sulfation of liver microsomal metabolites of skatole. The mean concentrations of skatole metabolite F-2 compared to controls were: 30 min, 301%; 60 min, 890%; 90 min, 964%, and did not differ from controls (P=274). Each data point represents the mean of duplicate assays performed for 3 pigs. PAPS is adenosine 3′-phosphate 5′-phosphosulfate. *Differs from control, P<0.05; **differs from control, P<0.01.

FIG. 10 shows the HPLC chromatogram of glucuronidation (A) and sulfation (B) of pro-MII (6-hydroxy-3-methylindole) in liver microsomes. Peaks 1 and 2, products of pro-MII glucuronidation; peak 3, product of pro-MII sulfation; IS, internal standard (indole-3-actetonitrile). The metabolites were detected by fluorescence, excitation wavelength, 285 nm and emission wavelength, 340 nm.

FIG. 11 is a graph showing the levels of skatole in fat versus the sultation of 5-hydroxyskatole.

FIG. 12 is a graph showing the inhibition of the sulfation of formation of 5-hydroxyskatole by DCNP (dichloronitrophenol) and PCP (pentachlorophenol). Each data point represents the mean and standard deviation of duplicate analysis on samples from three different pigs.

FIG. 13 is a graph showing plasma levels of skatole in control and group of uncastrated male pigs treated with ethanol from day 10 to day 50.

### DETAILED DESCRIPTION OF THE INVENTION

1. Methods of Determining Susceptibility to Boar Taint

As hereinbefore mentioned, the present inventors have determined that increased expression of CYP2E1 in the liver of pigs correlated with low levels of skatole in fat.

Accordingly, in one aspect the present invention provides a method for assessing the ability of a pig to metabolise skatole comprising (a) obtaining a sample from the pig and (b) detecting the levels of CYP2E1 in the sample wherein high levels of CYP2E1 indicates that the pig is a good skatole metaboliser. In another aspect, the present invention provides a method for determining the susceptibility of a male pig to developing boar taint comprising (a) obtaining a sample from the pig and (b) detecting the levels of CYP2E1 in the sample, wherein high levels of CYP2E1 indicates that the pig has a reduced susceptibility to developing boar taint.

The sample from the pig can be any sample wherein levels of CYP2E1 are correlated with levels of skatole in fat and thus boar taint. In a preferred embodiment, the sample is a liver sample or blood lymphocytes. The composition and activity of blood lymphocyte proteins, including CYP2E1, is closely related to that of the liver (Raucy et al., 1995; Yunjo et al., 1996) Levels of CYP2E1 can be measured using techniques known in the art including Western blotting as described in Example 1. Levels of CYP2E1 mRNA can also be measured by Northern analysis or quantitative PCR. Other methods include measuring the biological activity of the enzyme. For example, the activity of CYP2E1 can be measured by assaying its characteristic reactions, for example assaying for N-nitrosodimethylamine demethylase activity, aniline hydroxylase activity or p-nitrophenol hydroxylase activity as described in Xu et al., 1994. Alternatively, the activity of CYP2E1 can be measured by inhibiting the metabolism of skatole using known CYP2E1 inhibitors such as diallylsulfide and chlorozoxazone as described in Example 1, or 4-methylpyrazole as described in Halpert, et al.

The term “high levels of CYP2E1” means that the sample contains the same or higher levels of CYP2E1 than in a suitable control. Suitable controls include female pigs and male pigs that are known to have boar taint. When the control is a female pig “high levels of CYP2E1” means levels in the test pig are the same or higher than the control pig. When the control pig is a pig with boar taint, “high levels of CYP2E1” means levels; in the test pig are higher, preferably about 2-3 times higher than the level in a pig with boar taint. More preferably, the levels in the test pig are higher, preferably 2-3 times higher than the average level of CYP2E1 found in a group of pigs with boar taint. By “group” of pigs it is meant at least about 6 to about 10 male pigs.

The present inventors have also determined that increased expression of a thermostable phenol sulfotransferase correlated with low levels of skatole in fat. In another aspect, the present invention provides a method for assessing the ability of a pig to metabolise skatole comprising (a) obtaining a sample from the pig and (b) detecting the levels of thermostable phenol sulfotransferase in the sample wherein high levels of thermostable phenol sulfotransferase indicates that the pig is a good skatole metaboliser. In another aspect, the present invention provides a method for determining the susceptibility of a male pig to boar taint comprising (a) obtaining a sample from the pig and (b) detecting the levels of thermostable phenol sulfotransferase in the sample, wherein high levels of thermostable phenol sulfotransferase indicates that the pig has a reduced susceptibility to developing boar taint.

The sample from the pig can be any sample wherein levels of a thermostable phenol sulfotransferase are correlated with levels of skatole in fat and thus boar taint. In a preferred embodiment, the sample is a liver sample or blood platelets. A comparison of blood platelet phenol sulfotransferase activities with those in the liver of the same subjects showed that the levels of platelet thermostable phenol sulfotransferase activities were significantly correlated with levels of thermostable phenol sulfotransferase activity in the liver (r=0.79, n=14, P<00.001, Weinshilboum, 1990). Levels of a thermostable phenol sulfotransferase can be measured using techniques known in the art including Western blotting. Other methods include measuring the biological activity of the enzyme. For example, the activity of the sulfotransferase can be measured using 5-hydroxyskatole as a substrate as described in Example 2, Trial III, or using 2-naphthyl, p-nitrophenol or skatole as a substrate as described in Example 2.

The term “high levels of a thermostable phenol sulfotransferase” means that the sample contains the same or higher levels of a thermostable phenol sulfotransferase than in a suitable control. Suitable controls include female pigs and male pigs that are known to have boar taint. When the control is a female pig, “high levels of a thermostable phenol sulfotransferase” means levels in the rest pig are the same or higher than the control pig. When the control pig is a pig with boar taint, “high levels of a thermostable phenol sulfotransferase” means levels in the test pig are higher, preferably about 2 times higher, more preferably about 3-4 times higher than the level in a pig with boar taint, more preferably the levels in the test pig are higher, preferably about 2 times higher, more preferably about 3-4 times higher than the average level of thermostable phenol sulfotransferase found in a group of pigs with boar taint. By “group” of pigs it is meant at least about 6 to about 10 male pigs. When expressed in terms of enzyme activity the term “high levels of a thermostable phenol sulfotransferase” preferably means that a sample has an enzyme activity of greater than about 0.3, preferably greater than about 0.5, wherein activity is expressed as nm of 5-sulfatoxyskatole per minute per mg of protein.

In a further aspect, the present invention provides a method for assessing the ability of a pig to metabolise skatole comprising (a) obtaining a simple from the pig and (b) detecting the levels of glucuronyl transferase in the sample wherein low levels of glucuronyl transferase indicates that the pig is a good skatole metaboliser. In another aspect the present invention provides a method for determining the susceptibility of a male pig to boar taint comprising (a) obtaining a sample from the pig and (b) detecting the levels of glucuronyl transferase in the sample, wherein low levels of glucuronyl transferase indicates that the pig has a reduced susceptibility to developing boar taint.

The sample from the pig can be any sample wherein levels of glucuronyl transferase are corelated with levels of skatole in fat and thus boar taint. In a preferred embodiment, the sample is a liver sample. Levels of glucuronyl transferase can be measured using techniques known in the art including Western blotting. Other methods include measuring the biological activity of the enzyme, for example, as described in Example II.

The term “low levels of glucuronyl transferase” means that the sample contains the same or lower levels of glucuronyl transferase than in a suitable control. Suitable controls include female pigs and male pigs that are known to have boar taint. When the control is a female pig, “low levels of glucuronyl transferase” means levels in the test pig are the same or lower than the control pig. When the control pig is a pig with boar taint, “low levels of glucuronyl transferase” means levels in the rest pig are lower, preferably about 50% lower than the level of glucuronyl transferase found in a male pig with boar taint, more preferably the levels in the test pig are lower, preferably about 50% lower than the average level of glucuronyl transferase in a group of male pigs with boar taint. By “group” of pigs it is meant at least about 6 to about 10 male pigs.

Levels of one or more of CYP2E1, thermostable phenol sulfotransferase and/or glucuronyl transferase may also be measured in one sample obtained from the pig.

2. Methods of Enhancing Skatole Metabolism

As hereinbefore mentioned, the present invention relates to a method for preventing boar taint by enhancing the metabolism of skatole in a pig. The metabolism of skatole can be enhanced by altering the Phase I oxidation reactions carried out by cytochrome P450 and/or altering the Phase II conjugation reactions carried out by glucuronyl transferase and sulfotransferases.

Phase I Metabolism

In Phase I of skatole metabolism, the cytochrome P450 enzyme CYP2E1 (or P450IIE1) catalyzes the biotransformation of skatole to 6-hydroxy-skatole (pro-MII) and other metabolites including F-1 (indole-3-carbinol). The inventors have demonstrated that general inhibitors of cytochrome P450 and specific inhibitors of CYP2E1 reduced the production of skatole metabolites by liver microsomes. The inventors have shown that decreased expression of CYP2E1 in the liver of pigs is correlated with high skatole levels in the fat and increased expression of CYP2E1 in the liver of pigs is correlated with low skatole levels in the fat. The inventors have further shown that the induction of CYP2E1 by ethanol treatment (a known inducer of CYP2E1 activity) is effective in increasing skatole metabolism and clearance, and therefore is useful in treating boar taint in pig.

Accordingly, the present invention provides a method for reducing or preventing boar taint comprising enhancing the activity of CYP2E1 in a pig. The activity of the CYP2E1 enzyme can be enhanced by administering a substance (a) that increases the activity of the CYP2E1 enzyme; or (b) a substance that induces or increases the expression of the CYP2E1 gene. Substances that increase the activity of the CYP2E1 enzyme or induce or increase the expression of the CPY2E1 gene include substances that stabilize the protein and/or mRNA such as ethanol and acetone. CYP2E1 can also be induced by nutritional related conditions such as fasting and diabetes. The activity of the CYP2E1 may also be enhanced using gene therapy whereby a nucleic acid sequence encoding a CYP2E1 enzyme is introduced into a pig, either ex-vivo or in vivo. A nucleic acid sequence encoding a CYP2E1 enzyme may be obtained from GenBank Accession No. AB000885 or Swiss-Prot:P79383.

The inventors have also shown that the production of the skatole metabolites F-1 (indole-3-carbinol) and pro-MII (6-hydroxyskatole) were negatively correlated to skatole levels in fat.

Accordingly, the present invention also provides a method for detecting the capacity for boar taint comprising determining the production of F1 and/or pro-MII in a sample from a pig.

Phase II Metabolism

In Phase II of skatole metabolism, skatole and its metabolites (produced in Phase I) are conjugated by glucuronidation and sulfation in the liver. The inventors have shown that the formation and subsequent sulfation of the metabolite 6-hydroxyskatole (pro-MII) to 6-sulfatoxyskatole (MII) was related to low fat skatole levels, while high rates of glucuronidation were correlated with high skatole levels in fat. The inventors have also shown that the thermostable form of phenol sulfotransferase is involved in the sulfation of the skatole metabolite 5-hydroxyskatole. This suggests that high activity of thermostable phenol sulfotransferase and low glucuronyl transferase activity in the liver is necessary for effective clearance of skatole by the pig.

Accordingly, in another aspect, the present invention provides a method for reducing boar taint comprising enhancing the activity of a sulfotransferase in a pig. The activity of a sulfotransferase can be enhanced by administering (a) a substance that increases the activity of a sulfotransferase enzyme; or (b) a substance that induces or increases the expression of a sulfotransferase gene. In another embodiment, the activity of the sulfotransferase enzyme can also be enhanced by administering a nucleic acid sequence encoding a sulfotransferase enzyme into a pig, either ex vivo or in vivo.

Nucleic acid sequences encoding a sulfotransferases may be obtained from known sources. For example, the sequence of a human sulfotransferase may be obtained from GenBank Accession No. U52852, and the sequence of a guinea pig sulfotransferase may be obtained from GenBank accession on number L11117. Two families of sulfotransferases have been described in animals, the phenol sulfotransferase (PST) and the hydrocysteroid sulfotransferase. Five sulfotransferases have been isolated and cloned from human (Weishilboum et al., 1997). Three subfamilies of sulfotransferases exist in rats. Sulfotransferases have also been characterized in mice, guinea pigs, bovines as well as in plants (Matsui and Homma, 1994; Runge-Morris, 1997). Recently, the x-ray crystal structure of sulfotransferase has been reported (Negishi et al. 1998).

Human liver contains at least two distinct forms of PST which differ in their physical properties, substrate specificities, inhibitor sensitivities and regulation among individuals (Weinshilboum, 1990). The thermostable form of PST (TS-PST) catalyzes the sulfate conjugation of micromolar levels of p-nitrophenol and other simple phenols and is sensitive to inhibition by 2,6-dichloro-4-nitrophenol (DCNP) and pentachlorophenol (PCP); the thermolabile form (TL-PST) is active with micromolar levels of dopamine and other naturally occurring monoamines and is resistant to inhibition by DCNP and PCP (Hernández et al., 1991; Rein et al., 1982). Dopamine is the substrate that has been used most often to measure TL-PST activity while p-nitrophenol is the model substrate for the measurement of TS-PST activity (Weinshilboum, 1990).

Estrogens decrease while androgens increase sulfotransferase activity in rats (Matsui and Homma, 1994), while there are no pronounced differences in sulfotransferase between sexes in humans (Tamellini et al., 1991). Genetic polymorphisms for sulfotransferase have been reported for several mammalian species (Weishilboum et al., 1997) and may also exist in the pig. The significant interindividual variability (Pacifici et al., 1994) and genetic polymorphism of sulfotransferases likely explains why some pigs are more susceptible to developing boar taint.

In a further aspect, the present invention provides a method for reducing boar taint comprising inhibiting the activity of a glucuronyl transferase in a pig. The activity of a glucuronyl transferase enzyme may be inhibited by administering (a) a substance that inhibits the activity of the glucuronyl transferase enzyme; or (b) a substance that inhibits the expression of the glucuronyl transferase gene. Substances that inhibit the activity of a glucuronyl transferase enzyme include antibodies to the enzyme. Antibodies capable of binding to a glucuronyl transferase enzyme can be prepared using techniques known in the art. For example, by using a peptide or the entire enzyme, polyclonal antisera or monoclonal antibodies can be made using standard methods. A mammal, (e.g., a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g., the hybridoma technique originally developed by Kohler and Milstein (Nature 256, 495-497 (1975)) as well as other techniques such as the human B-cell hybridoma technique (Kozbor et al., Immunol. Today 4, 72 (1983)), the EBV-hybridoma technique to produce human monoclonal antibodies (Cole et al., Monoclonal Antibodies in Cancer Therapy (1985) Allen R. Bliss, Inc., pages 77-96), and screening of combinatorial antibody libraries (Huse et al., Science 246, 1275 (1989)). Hybridoma cells can be screened immunologically for production of antibodies specifically reactive with the peptide and the monoclonal antibodies can be isolated. Therefore, the invention also contemplates hybridoma cells secreting monoclonal antibodies with specificity for a glucuronyl transferase of the invention.

The term “antibody” as used herein is intended to include fragments thereof which also specifically react with a protein, of the invention, or peptide thereof. Antibodies can be fragmented using conventional techniques and the fragments screened for utility in the same manner as described above. For example, F(ab′)₂ fragments can be generated by treating antibody with pepsin. The resulting F(ab′)₂ fragment can be treated to reduce disulfide bridges to produce Fab′ fragments.

Chimeric antibody derivatives, i.e., antibody molecules that combine a non-porcine variable region and a porcine constant region are also contemplated within the scope of the invention. Chimeric antibody molecules can include, for example, the antigen binding domain from an antibody of a mouse, rat, or other species, with porcine constant regions. Conventional methods may be used to make chimeric antibodies containing the immunoglobulin variable region which recognizes a protein of the invention (See, for example, Morrison et al., Proc. Natl Acad. Sci. U.S.A. 81,6851 (1985); Takeda et al., Nature 314, 452 (1985), Cabilly et al., U.S. Pat. No. 4,816,567; Boss et al., U.S. Pat. No. 4,816,397; Tanaguchi et al., European Patent Publication EP171496; European Patent Publication 0173494, United Kingdom patent GB 2177096B).

Substances that inhibit the expression of the glucuronyl transferase gene include antisense nucleic acid sequences that are complimentary to a sequence of the glucuronyl transferase gene. The antisense sequences of the present invention may be ribonucleic or deoxyribonucleic acids and may contain naturally occurring bases including adenine, guanine, cytcosine, thymidine and uracil. The antisense sequences may also contain modified bases such as xanthine, hypoxanthine, 2-aminoadenine, 6-methyl, 2-propyl and other alkyl adenines, 5-halo uracil, 5halo cytosine, 6-aza uracil, 6-aza cytosine and 6-aza tymine, pseudo uracil, 4thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thiolalkyl guanines 8-hydroxyl guanine and other 8-substituted guarines, other aza and deaza uracils, thymidines, cytosines, adenines, or guanines, 5-trifluoromethyl uracil and 5-trifluoro cytosine. In addition, the antisense sequences of the invention may contain modified phosphorous, oxygen heteroatoms in the phosphate backbone, short chain alkyl or cycloalkyl intersugar linkages or short chain heteroatomic or heterocyclic intersugar linkages. For example, the antisense oligonucleotides may contain phosphorothioates, phosphotriesters, methyl phosphonates, and phosphorodithioates. In an embodiment of the invention there are phosphorothioate bonds links between the four to six 3′-terminus bases. In another embodiment phosphorothioate bonds link all the nucleotides.

The antisense sequences of the invention may also comprise nucleotide analogs that may be better suited as therapeutic or experimental reagents. An example of an oligonucleotide analogue is a peptide nucleic acid (PNA) wherein the deoxyribose (or ribose) phosphate backbone in the DNA (or RNA), is replaced with a polyamide backbone which is similar to that found in peptides (P. E. Nielsen, et al Science 1991, 254, 1497).

3. Screening Methods

As hereinbefore mentioned, the present invention provides a method of screening for a substance that regulates skatole metabolism in a pig. Substances that regulate skatole metabolism include substances that modulate the Phase I oxidation or Phase II conjugation reactions involved in skatole metabolism. Preferably, the substances enhance the activity or expression of CYP2E1 or a thermostable phenol sulfotransferase or inhibit the activity or expression of a glucoronyl transferase and are useful in reducing boar taint.

Substances Which Modulate Enzyme Activity

In one aspect, the present invention provides a method of screening for a substance that enhances the activity of CYP2E1 or a thermostable phenol sulfotransferase and/or inhibits the activity of a glucuronyl transferase.

(a) CYP2E1

In one embodiment of the invention, a method is provided for screening for a substance that enhances skatole metabolism in a pig by enhancing CYP2E1 activity comprising the steps of:

(a) reacting a substrate of CYP2E1 and CYP2E1, in the presence of a rest substance, under conditions such that CYP2E1 is capable of converting the substrate into a reaction product;

(b) assaying for reaction product, unreacted substrate or unreacted CYP2E1;

(c) comparing to controls to determine if the test substance selectively enhances CYP2E1 activity and thereby is capable of enhancing skatole metabolism in a pig.

Substrates of CYP2E1 which may be used in the method of the invention for example include skatole and analogs and derivatives thereof. The corresponding reaction products for skatole are pro-MII and F-1.

Levels of CYP2E1 can be measured using techniques known in the art including Western blotting as described in Example 1. Levels of CYP2E1 mRNA can also be measured by Northern analysis or quantitative PCR. Other methods include measuring the biological activity of the enzyme. For example, the activity of CYP2E1 can be measured by estimating its characteristic reactions for example assaying for N-nitrosodimethylamine demethylase activity, aniline hydroxylase activity or p-nitrophenol hydroxylase activity as described in Xu et al., 1994. Alternatively, the activity of CYP2E1 can be measured by inhibiting the metabolism of skatole using known CYP2E1 inhibitors such as diallylsulfide and chlorozoxazone as described in Example I, or 4-methylpyrazole as described in Halpert et al., 1994.

(b) Sulfotransferase

In another embodiment of the invention, a method is provided for screening for a substance that enhances skatole metabolism in a pig by enhancing sulfotransferase activity comprising the steps of.

(a) reacting a substrate of sulfotransferase and sulfotransferase, in the presence of a test substance, under conditions such that sulfotransferase is capable of converting the substrate into a reaction product;

(b) assaying for reaction product, unreacted substrate or unreacted sulfotransferase;

(c) comparing to controls to determine if the test substance selectively enhances sulfotransferase activity and thereby is capable of enhancing skatole metabolism in a pig.

Substrates of sulfotransferase which may be used in the method of the invention for example include pro-MII and analogs and derivatives thereof, and 2-naphthol. The corresponding reaction product for pro-MII is MII.

The induction of sulfotransferase activity can be measured using a variety of techniques known in the art. For example levels of a thermostable phenol sulfotransferase can be measured using Western blotting. Other methods include measuring the biological activity of the enzyme. For example, the activity of the sulfotransferase can be measured using 5-hydroxyskatole as a substrate as described in Example 2, Trial III, or using 2-naphthyl, p-nitrophenol or skatole as a substrate as described in Example 2.

(c) Glucuronyl Transferase

In a further embodiment of the invention, a method is provided for screening for a substance that enhances skatole metabolism in a pig by inhibiting glucuronyl transferase activity comprising the steps of:

(a) reacting a substrate of glucuronyl transferase and glucuronyl transferase, in the presence of a test substance, under conditions such that glucuronyl transferase is capable of converting the substrate into a reaction product;

(b) assaying for reaction product, unreacted substrate or unreacted glucuronyl transferase;

(c) comparing to controls to determine if the test substance selectively inhibits glucuronyl transferase activity and thereby is capable of inhibiting skatole metabolism in a pig.

Substrates of glucuronyl transferase which may be used in the method of the invention for example include pro-MII and analogs and derivatives thereof, 2-naphthol and p-nitrophenol The corresponding reaction product for pro-MII is MII.

The inhibition of glucuronyl transferase activity can be measured using a variety of techniques including measuring the levels of the glucuronyl transferase protein or mRNA or by testing for glucuronyl transferase activity, for example, as described in Example 2.

The CYP2E1, sulfotransferase and glucuronyl transferase enzymes used in the method of the invention may be obtained from natural, recombinant, or commercial sources. Cells or liver microsomes expressing the enzymes may also be used in the method.

Conditions which permit the formation of a reaction product may be selected having regard to factors such as the nature and amounts of the test substance and the substrate.

The reaction product, unreacted substrate, or unreacted enzyme; may be isolated by conventional isolation techniques, for example, salting out, chromatography, electrophoresis, gel filtration, fractionation, absorption, polyacrylamide gel electrophoresis, agglutination, or combinations thereof. To facilitate the assay of the reaction product, unreacted substrate, or unreacted enzyme; antibody against the reaction product or the substance, or a labelled enzyme or substrate, or a labelled substance may be utilized. Antibodies, enzyme, substrate, or the substance may be labelled with a detectable marker such as a radioactive label, antigens that are recognized by a specific labelled antibody, fluorescent compounds, enzymes, antibodies specific for a labelled antigen, and chemiluminescent compounds.

The substrate used in the method of the invention may be insolubilized. For example it may be bound to a suitable carrier. Examples of suitable carriers are agarose, cellulose, dextran, Sephadex, Sepharose, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, beads, disc, sphere etc. The insolubilized enzyme, substrate, or substance may be prepared by reacting the material with a suitable insoluble carrier using known chemical or physical methods, for example, cyanogen bromide coupling.

Substances which Modulate Gene Expression

In another aspect, the present invention includes a method for screening for a substance that enhances skatole metabolism by modulating the transcription or translation of an enzyme involved in skatole metabolism.

(a) CYP2E1

In one embodiment of the invention, a method is provided for screening for a substance that enhances skatole metabolism by enhancing transcription and/or translation of the gene encoding CYP2E1 comprising the steps of:

(a) culturing a host cell comprising a nucleic acid molecule containing a nuclei acid sequence encoding CYP2E1 and the necessary elements for the transcription or translation of the nucleic acid sequence, and optionally a reporter gene, in the presence of a test substance; and

(b) comparing the level of expression of CYP2E1, or the expression of the protein encoded by the reporter gene with a control cell transfected with a nucleic acid molecule in the absence of the test substance.

(b) Sulfotransferase

In another embodiment of the invention, a method is provided for screening for a substance that enhances skatole metabolism by enhancing transcription and/or translation of the gene encoding sulfotransferase comprising the steps of:

(a) culturing a host cell comprising a nucleic acid molecule containing a nuclei acid sequence encoding sulfotransferase and the necessary elements for the transcription or translation of the nucleic acid sequence, and optionally a reporter gene, in the presence of a test substance; and

(b) comparing the level of expression of sulfotransferase, or the expression of the protein encoded by the reporter gene with a control cell transfected with a nucleic acid molecule in the absence of the test substance.

(c) Glucuronyl Transferase

In a further embodiment of the invention, a method is provided for screening for a substance that enhances skatole metabolism by inhibiting transcription and/or translation of the gene encoding glucuronyl transferase comprising the steps of:

(a) culturing a host cell comprising a nucleic acid molecule containing a nuclei acid sequence encoding glucuronyl transferase and the necessary elements for the transcription or translation of the nucleic acid sequence, and optionally a reporter gene, in the presence of a test substance; and

(b) comparing the level of expression of glucuronyl transferase, or the expression of the protein encoded by the reporter gene with a control cell transfected with a nucleic acid molecule in the absence of the rest substance.

A host cell for use in the method of the invention may be prepared by transfecting a suitable host with a nucleic acid molecule comprising a nucleic acid sequence encoding the appropriate enzyme. Suitable transcription and translation elements may be derived from a variety of sources, including bacterial, fungal, viral, mammalian, or insect genes. Selection of appropriate transcription and translation elements is dependent on the host cell chosen, and may be readily accomplished by one of ordinary skill in the art. Examples of such elements include: a transcriptional promoter and enhancer or RNA polymerase binding sequence, a ribosomal binding sequence, including a translation initiation signal. Additionally, depending on the host cell chosen and the vector employed, other genetic elements, such as an origin of replication, additional DNA restriction sites, enhancers, and sequences conferring inducibility of transcription may be incorporated into the expression vector. It will also be appreciated that the necessary transcription and translation elements may be supplied by the native gene of the enzyme and/or its flanking sequences.

Examples of reporter genes are genes encoding a protein such as β-galactosidase, chloramphenicol acetyltransferase, firefly luciferase, or an immunoglobulin or portion thereof such as the Fc portion of an immunoglobulin, preferably IgG. Transcription of the reporter gene is monitored by changes in the concentration of the reporter protein such as β-galactosidase, chloramphenicol aceryltransferase, or firefly luciferase. This makes it possible to visualize and assay for expression of the enzyme and in particular to determine the effect of a substance on expression of enzyme.

Suitable host cells include a wide variety of prokaryotic and eukaryotic host cells, including bacterial, mammalian, yeast or other fungi, viral, plant, or insect cells. Protocols for the transfection of host cells are well known in the art (see, Sambrook et al. Molecular Cloning A Laboratory Manual, 2nd edition, Cold Spring Harbor Laboratory Press, 1989, which is incorporated herein by reference). Host cells which are commercially available may also be used in the method of the invention. For example, the h2A3 and h2B6 cell lines available from Gentest Corporation are suitable for the screening methods of the invention.

4. Compositions

Substances which enhance skatole metabolism described in detail herein or substances identified using the methods of the invention which selectively enhance CYP2E1 or sulfotransferase activity or inhibit glucuronyl transferase activity (including antibodies or antisense sequences) may be incorporated into pharmaceutical compositions. Therefore, the invention provides a pharmaceutical composition for use in reducing boar taint comprising an effective amount of one or more substances which enhance skatole metabolism and/or a pharmaceutically acceptable carrier, diluent, or excipient. In one embodiment, the present invention provides a pharmaceutical composition comprising an effective amount of the substance which is selected from the group consisting of

(a) a substance that increases the activity of the CYP2E1 enzyme;

(b) a substance that induces or increases the expression of the CYP2E1 gene;

(c) a substance that increases the activity of the sulfotransferase enzyme;

(d) a substance that induces or increases the expression of the sulfotransferase gene;

(e) a substance that decreases the activity of the glucuronyl transferase enzyme; and

(f) a substance that reduces or decreases the expression of the glucuronyl transferase gene.

The substances for the present invention can be administered for oral, topical, rectal, parenteral, local, inhalant or intracerebral use. Preferably, the active substances are administered orally (in the food or drink) or as an injectable formulation.

In the methods of the present invention, the substances described in detail herein and identified using the method of the invention form the active ingredient, and are typically administered in admixture with suitable pharmaceutical diluents, excipients, or carriers suitably selected witch respect to the intended form of administration, that is, oral tablets, capsules, elixirs, syrups and the like, consistent with conventional vetrinary practices.

For example, for oral administration the active ingredients may be prepared in the form of a tablet or capsule for inclusion in the food or drink. In such a case, the active substances can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral active substances can be combined with any oral, non-toxic, pharmaceutically acceptable inert carrier such as ethanol, glycerol, water, and the like. Suitable binders, lubricants, disintegrating agents, and coloring agents can also be incorporated into the dosage form if desired or necessary. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Suitable lubricants used in these dosage forms include sodium oleate, sodium stearate, magnesium stearate, sodium benzoate, sodium acetate, sodium chloride, and the like. Examples of disintegrators include starch, methyl cellulose, agar, bentonite, xanthan gum, and the like.

Gelatin capsules may contain the active substance and powdered carriers, such as lactose, starch, cellulose derivatives, magnesium stearate, stearic acid, and the like. Similar carriers and diluents may be used to make compressed tablets. Tablets and capsules can be manufactured as sustained release products to provide for continuous release of active ingredients over a period of time. Compressed tablets can be sugar coated or film coated to mask any unpleasant taste and protect the tablet from the atmosphere, or enteric coated for selective disintegration in the gastrointestinal tract. Liquid dosage forms for oral administration may contain coloring and flavoring agents to increase acceptance.

Water, a suitable oil, saline, aqueous dextrose, and related sugar solutions and glycols such as propylene glycol or polyethylene glycols, may be used as carriers for parenteral solutions. Such solutions also preferably contain a water soluble salt of the active ingredient, suitable stabilizing agents, and if necessary, buffer substances. Suitable stabilizing agents include antioxidizing agents such as sodium bisulfate, sodium sulfite, or ascorbic acid, either alone or combined, citric acid and its salts and sodium EDTA. Parenteral solutions may also contain preservatives, such as benzalkonium chloride, methyl- or propyl-paraben, and chlorobutanol.

The substances described in detail herein and identified using the methods of the invention can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Substances described in detail herein and identified using the methods of the invention may also be coupled with soluble polymers which are targetable drug carriers. Examples of such polymers include polyvinylpyrrolidone, pyran copolymer, polyhydroxypropyl-methacrylamidephenol, polyhydroxyethyl-aspartamidephenol, or polyethyleneoxide-polylysine substituted with palmitoyl residues. The substances may also be coupled to biodegradable polymers useful in achieving controlled release of a drug. Suitable polymers include polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Suitable pharmaceutical carriers and methods of preparing pharmaceutical dosage forms are described in Remington's Pharmaceutical Sciences Mack Publishing Company, a standard reference text in this field.

More than one substance described in detail herein or identified using the methods of the invention may be used to enhance metabolism of skatole. In such cases the substances can be administered by any conventional means available for the use in conjunction with pharmaceuticals, either as individual separate dosage units administered simultaneously or concurrently, or in a physical combination of each component therapeutic agent in a single or combined dosage unit. The active agents can be administered alone, but are generally administered with a pharmaceutical carrier selected on the basis of the chosen route of administration and standard pharmaceutical practice as described herein

5. Genetic Screening

The present invention further includes the identification of polymorphisms in genes encoding the enzymes responsible for skatole metabolism in a pig including CYP2E1 thermostable phenol sulfotransferases and glucuronyl transferase as described in detail hereinabove. The identification of genes that encode these enzymes from pigs that are high skatole metabolizers (and hence have a low incidence of low boar taint) can be used to develop lines of pigs that have a low incidence of boar taint. In addition, the identification of these genes can be used as markers for identifying pigs that are predisposed to having a low incidence of boar taint.

Accordingly, the present invention provides a method for producing pigs which have a lower incidence of boar taint comprising selecting pigs that express high levels of CYP2E1 and/or thermostable phenol sulfotransferase and/or low levels of glucuronyl transferase and breeding the selected pigs.

Transgenic pigs may also be prepared which produce high levels of CYP2E1 and/or thermostable phenol sulfotransferase and/or low levels of glucuronyl transferase. The transgenic pigs may be prepared using conventional techniques. For example, a recombinant molecule may be used to introduce (a) a gene encoding CYP2E1 or (b) a gene encoding a thermostable phenol sulfotransferase or (c) an antisense nucleic acid molecule complimentary to a glucuronyl transferase. Such recombinant constructs may be introduced into cells such as embryonic stem cells, by a technique such as transfection, electroporation, injection, etc. Cells which show high levels of CYP2E1 and/or sulfotransferase and/or low levels of glucuronyl transferase may be identified for example by Southern Blotting, Northern Blotting, or by other methods known in the art. Such cells may then be fused to embryonic stem cells to generate transgenic animals. Germline transmission of the mutation may be achieved by, for example, aggregating the embryonic stem cells with early stage embryos, such as 8 cell embryos, transferring the resulting blastocysts into recipient females in vitro, and generating germline transmission of the resulting aggregation chimeras. Such a transgenic pig may be mated with pigs having a similar phenotype i.e. producing high levels of CYP2E1 and/or sulfotransferase and/or low levels of glucuronyl transferase to produce animals having a low incidence of boar taint. The following non-limiting examples are illustrative of the present invention:

### EXAMPLES

### Example 1

### Involvement of Cytochrome P4502E1 in Oxidation Reactions in Skatole Metabolism

Materials and Methods

Chemicals

Skatole (3-methylindole), indole-3-acetonitrile, NADH (disodium salt), NADPH (tetrasodium salt), diallyl sulfide (DAS), chlorzoxazone and metyrapone were purchased from Sigma Chemical (St. Louis, Mo.) SKF 525A hydrochloride (Proadifen) was purchased from Research Biochemical Inc (Natick, Mass.). 6-Hydroxy-3-methylindole (pro-MII) and 3-hydroxy-3-methyloxindole (MIII) were kindly provided by Jens Hansen Moler of the Danish Meat Research Institute, Roskilde, Denmark.

Animals

Intact male pigs obtained by back-crossing F3 European Wild Pig×Swedish Yorkshire pigs with Swedish Yorkshire sows (Anderson et al., 1994; Squires and Lundström, 1997) were used. They were slaughtered at approximately 108 kg live weight. Another group of animals consisting of 30 purebred Swedish Yorkshire entire male pigs and 8 female pigs was also included. Blood samples were also collected from about half of the male pigs. Liver samples were frozen in liquid nitrogen within 30 min after exsanguination and kept at −70° C. until analysis, while plasma and fat were kept in −20° C. Backfat samples were analyzed for skatole concentration with a colorimetric assay (Mortensen and Sorensen, 1984). Liver samples were used for subsequent preparation of microsomes and analysis of levels of CYP2E1 and for analysis of skatole metabolism. On the basis of a preliminary analysis of skatole metabolism by liver microsomes, 9 pigs with sufficient level of production of skatole metabolites for HPLC analysis were chosen. Concentrations of CYP2E1 in the liver of these pigs were medium to high (mean, 62.1; SD, 32.4 of the arbitrary units used by Squires and Lundström, 1997), while concentrations of skatole in the backfat were low (mean, 0.14; SD, 00.04 ppm).

Preparation of Microsomes

Liver microsomes were prepared as previously described by Meadus and Squires (1995)

Enzyme Assays

The metabolism of skatole was assayed in a medium containing 50 mM Tris-HCL buffer, pH 7.4, 10 mM potassium phosphate 0.1 mM EDTA, 20% glycerol, 0.01 mM skatole and 1 mg/mL microsomal protein in a total volume of 2 mL. The reactions were started by addition of NADH and NADPH to 1 mM final concentration of each after 3 min of preincubation at 37° C. After 30 min at 37° C., the reactions were terminated by addition of 2 mL of ice-cold 0.2 M ammonium acetate buffer, pH 50.0.

Those incubations with no inhibitor added were regarded as controls. The effect of different P450 inhibitors on skatole metabolism was evaluated under the same conditions by including different concentrations of P450 inhibitors into the medium before preincubation. The concentrations of the inhibitors ranged from 0.025 to 0.2 mM of SKF 525A, diallyl sulfide (DAS) or chlorzoxazone, or 0.025 to 0.1 mM of metyrapone. At higher inhibitor concentrations up to 0.4 mM, no significant changes in the formation of metabolites or skatole disappearance were observed.

Each assay was performed in duplicate. The assays of formation of skatole metabolites over time were performed with microsomes from 2 different randomly chosen pigs. The control incubations and the incubations with P450 inhibitors were performed with microsomes from 3 different randomly chosen pigs.

Analysis of skatole metabolites

The procedures for extraction and analysis of skatole metabolites by HPLC were based on the method described by Bæk et al. (1995). After adding ammonium acetate buffer, 5 μL of 0.01 mg/mL indole-3-acetonitrile was added to the reaction mixture as an internal standard. The mixture was then passed through a solid phase extraction tC18 cartridge (Waters Co., Milford, Mass.) previously conditioned with 2 mL of methanol and then 2 mL of water. The column was washed with 1 mL of water and skatole metabolites were eluted with 2×0.5 mL of acetonitrile. The acetonitrile was removed under a gentle stream of nitrogen at 40° C. and the metabolites were resuspended in 200 μl of acetonitrile for HPLC analysis. The HPLC equipment included a Spectra-Physics (San Jose, Calif., USA) SP8800 pump, SP8800 autosampler and SP4290 integrator, and a Phenomenex (Torrance, Calif., USA) Prodigy ODS-2 C18, 50×4.6 mm, 5 μm column. The analysis was performed using 3 buffers, A—acetonitrile; 0.01 mM potassium phosphate buffer, pH 3.9 (10:90), B—acetonitrile; water (90:10) and C—acetonitrile, with the following gradient: 0 min—A, 100%, 6 min—A, 75%, B, 25%; 15 min—A, 75%, B, 25%; 18 min—A, 20%, B, 80%; 20 min—B, 100%; 23 min—C, 100%; 25 min—C, 100%; 26 min—A, 100%; 30 min A, 100%. Injection volume was 100 μl. Skatole metabolites were detected simultaneously using both a UV detector set at 250 nm (Spectra 100, Spectra-Physics, San Jose, Calif., USA) and fluorescence detector (Shimadzu RF-535; Shimadzu Co., Kyoto, Japan), excitation wavelength of 285 nm and emission wavelength of 340 nm.

Determination of CYP2E1 by Western Analysis

Levels of the liver enzyme CYP2E1 were determined using a Western blotting kit for CYP2E1 from rat (Amersham International plc, Buckinghamshire, England). Levels of CYP2E1 were estimated from the intensity of the bands on the autoradiographs using an ISI-1000 digital imaging system (Alpha Immunotech Corp., San leandro, Calif. 94577)

Statistical analysis

The effect of different concentrations of P450 inhibitors on the concentration of skatole and its metabolites in microsomal incubations was analyzed with Statistical Analysis System (SAS Institute, 1995) using GLM procedure. For the purpose of the analysis, peak areas of the metabolites in control incubations were considered as 100%. Peak areas of the metabolites in the incubations with inhibitors were transformed to % values of those for controls. The statistical model included the effect of the concentration of inhibitor and the effect of animal.

Results

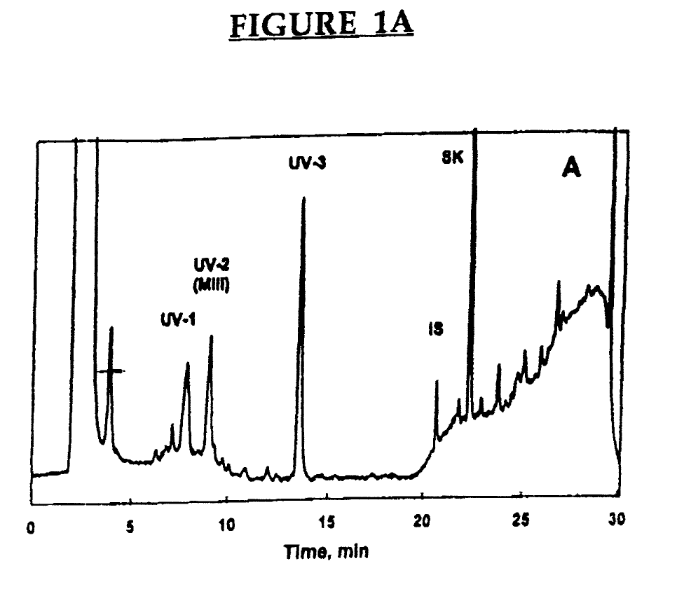

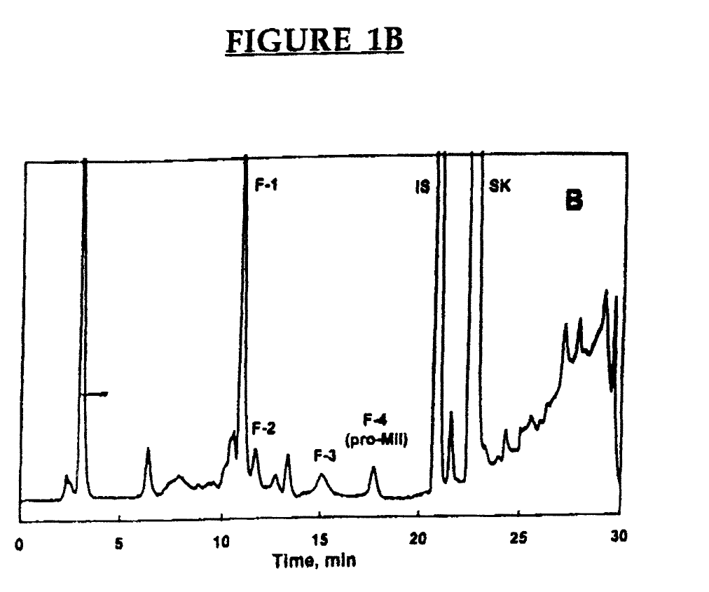

A HPLC profile of microsomal metabolites of skatole is shown in FIG. 1. Several microsomal metabolites of skatole were found, the peaks F-4 and UV-2 were identified as 6-hydroxyskatole (pro-MII), which is a precursor of its sulfated form, 6-sulfatoxyskatole (MII), and 3-hydroxy-3-methyloxindole (MII), respectively, using standards. Other metabolites include UV-1 (3-hydroxy-3-methylindolenine), UV-3 (3-methyloxindole), F-1 (indole-3-carbinol), F-3 was not identified.

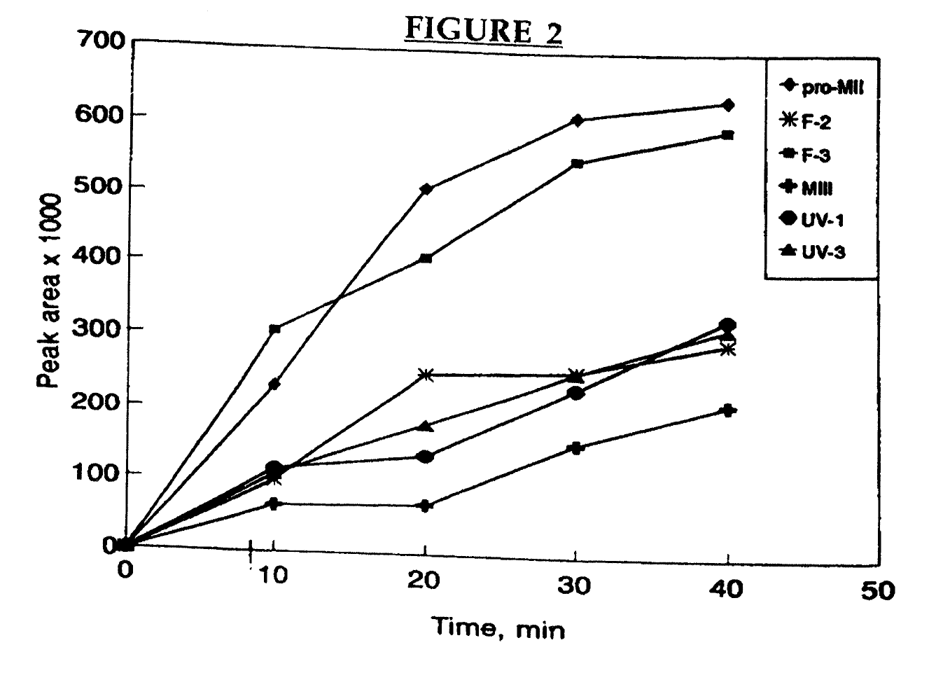

FIG. 2 shows that the production of skatole metabolites by liver microsomes was approximately linear for 30 min and this period of time was chosen for subsequent assays using cytochrome P450 inhibitors. Metabolite F-1 is not presented in FIG. 2 because of its much larger peak area than that of the other metabolites. Its synthesis pattern over time followed that of the other metabolites.

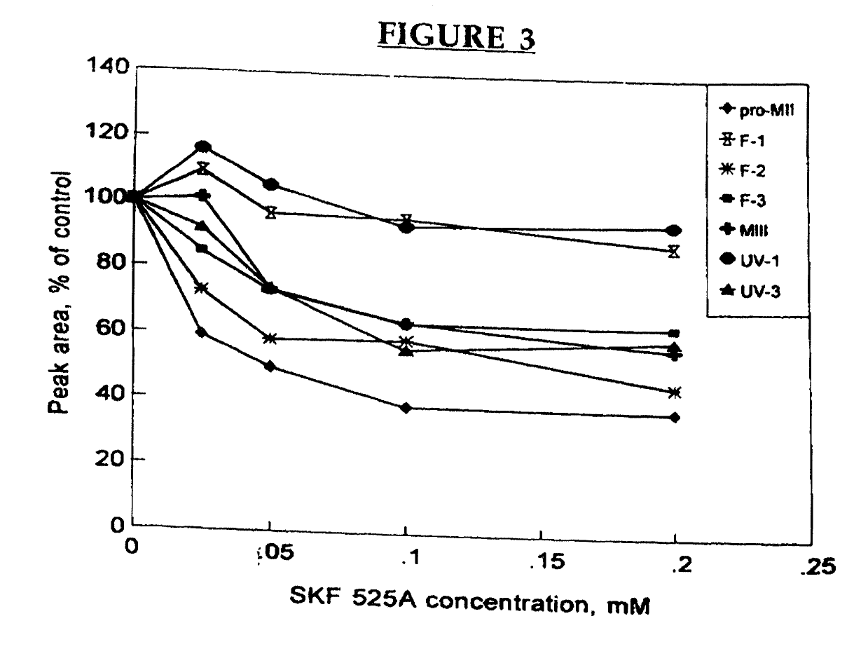

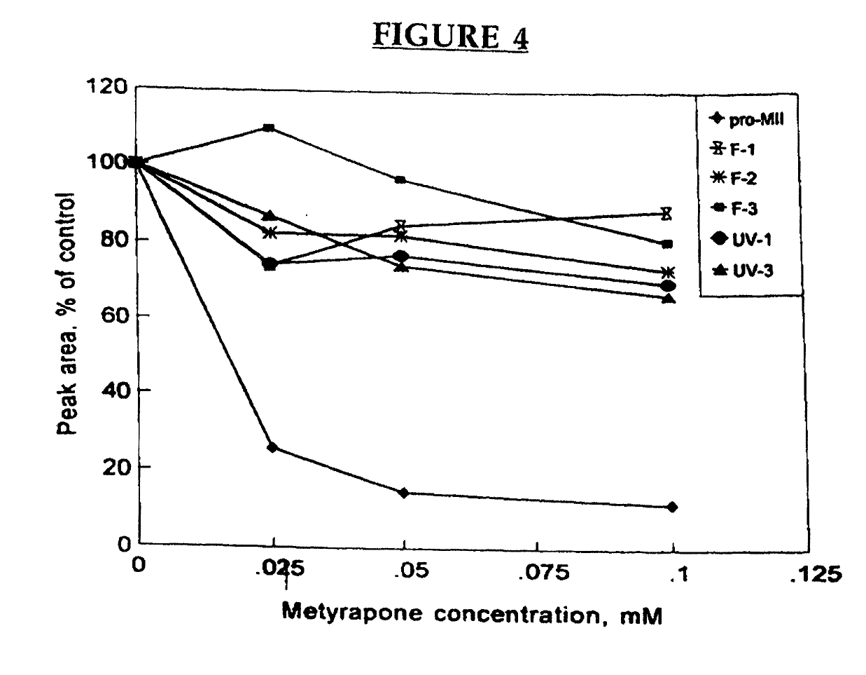

The microsomal assays which included addition of SKF 525A (FIG. 3) or metyrapone (FIG. 4) indicated a significant effect (P=0.001) on pro-MII synthesis. MIII could not be separated by HPLC from an unidentified metabolite of metyrapone, and therefore it was not possible to quantify MIII formation. The synthesis of pro-MII was decreased with 0.025 mM of added inhibitor (P<0.05; Table 1), decreasing to 38.2% with 0.2 mM of SKF 525A and 11.6% with 0.1 mM of metyrapone compared to controls. Formation of MIII was inhibited by SKF 525A (P<0.001). The effect of metyrapone on MII could not be evaluated, because a metabolite of metyrapone co-eluted with MIII in the HPLC analysis. Both inhibitors reduced formation of UV-3. SKF 525A also reduced formation of F-2 and F-3 (P<0.05). The amount of skatole remaining in the reaction mixture after incubation was higher for the assays with metyrapone than for control (P=0.008), and not affected by SKF 525A (P=0.613).

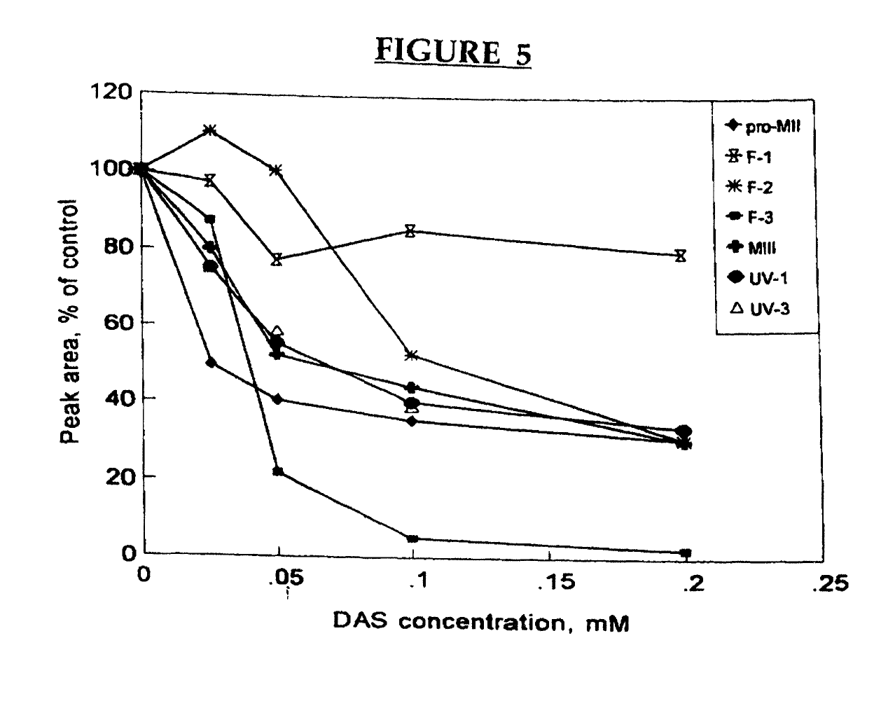

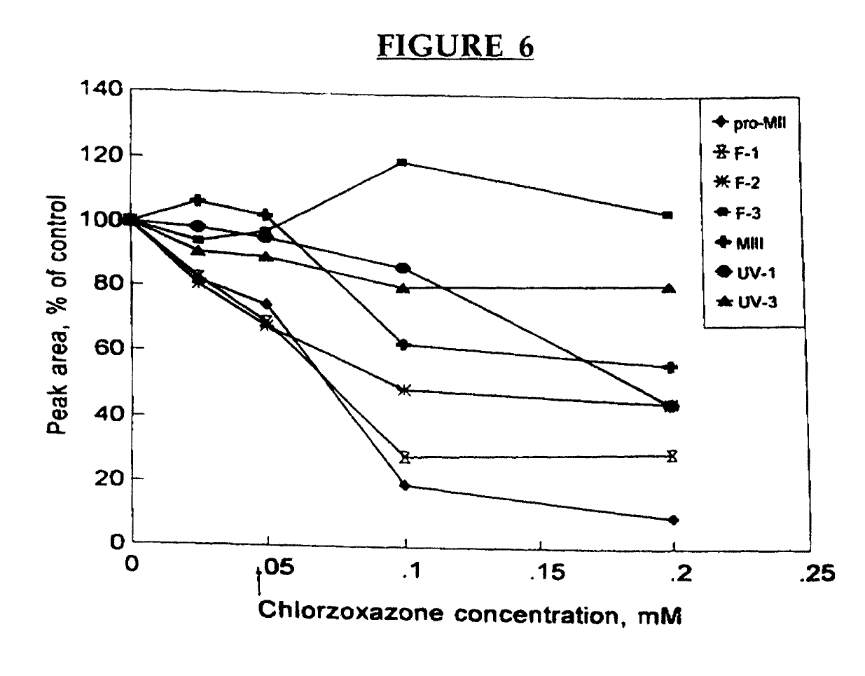

The specific inhibitors of CYP2E1 were more effective in reducing skatole metabolism and formation of its metabolites than was SKF 525A and metyrapone. The synthesis of pro-MII was significantly reduced with 0.025 mM of DAS or chlorzoxazone, while at 0.2 mM of these inhibitors the synthesis of MIII was reduced to 30.9% and 9.7%, respectively. Both inhibitors reduced MII synthesis and skatole disappearance (Table 1). DAS also inhibited the formation of all the other skatole metabolites (P=0.001), except for F-1 (P=0.336; FIG. 5). Chlorzoxazone inhibited the formation of all skatole metabolites (P<0.05), except for F-3 (P=0.680; FIG. 6).

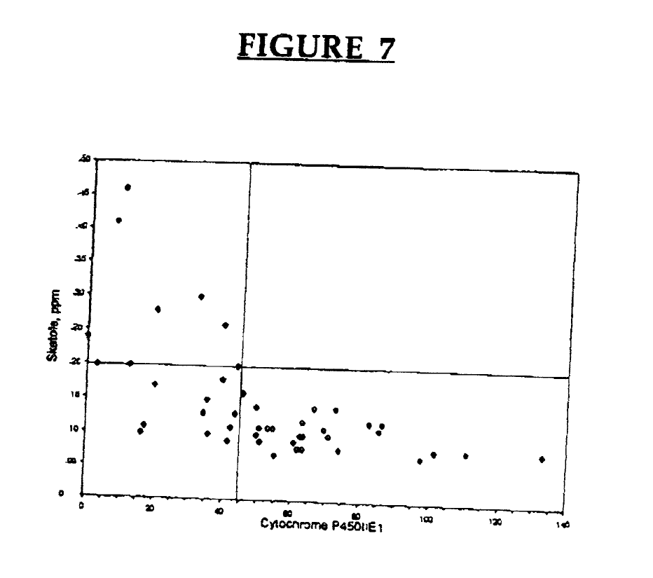

A plot of the levels of skatole in fat against the levels of CYP2E1 in liver (FIG. 7) illustrates that animals with high CYP2E1 levels had low skatole levels in fat. However, pigs with CYP2E1 levels below 45 (the lowest level of CYP2E1 found in male and female Yorkshire pigs) had both low and high skatole levels. No similar relationship was found between 5α-androstenone and CYP2E1 using the 1 ppm threshold value for 5α-androstenone, but pigs with extremely high 5α-androstenone concentrations exceeding 3 ppm also had CYP2E1 concentrations below 45 (4/32 in the Wild Pig crosses).

The correlations among the substances varied between the two groups of male pigs (Table 2). In the Wild Pig crosses, skatole was correlated with androstenone, cytochrome P450IIE1, estrone sulfate and the skatole metabolite MIII, while all correlations between skatole and the other substances were non-significant in the Yorkshire pigs. In addition, levels of cytochrome P450IIE1 were not correlated with either MII or MIII or the MII/MIII ratio in any of the groups of animal.

Discussion

In this study, the involvement of the liver enzyme system, cytochrome P450, in the biodegradation of skatole to pro-MII and other metabolites was evaluated.

In this study, microsomal incubations were conducted using standard procedures for evaluating P450 activity, using NADPH and NADPH as electron donors. These incubations revealed formation of several skatole metabolites, including pro-MII and MIII detected by either fluorescence or UV absorbance after separation on HPLC (FIG. 1 and 2). Besides pip, MIII has been also found in mouse urine (Skiles et al., 1989) and, in a conjugated form, in goat urine (Smith et al, 1993). Pro-MII or MII has not yet been reported in other species than pigs.

The formation of MII in microsomal incubations was inhibited by both classical inhibitors of cytochrome P450, metyrapone and SKF 252A. These inhibitors form ligand complexes with cytochrome P450, causing spectral changes of the enzymes (Netter 1980). Metyrapone forms complexes with variety of P450 isozymes including P4502E1 (Knecht et al., 1993), by binding competitively to P450 iron binding site II preventing oxidation of a substrate (Netter, 1980). However, the affinity of metyrapone towards different forms of P450 is substantially different (Roos et al., 1993) and some of the isozymes are not affected by this compound (Ryan et al., 1984). These could be the reasons for a strong inhibitory effect of metyrapone on pro-MIII formation, reduction of UV-3 formation and skatole disappearance but no effect on the formation of the other metabolites. SKF 252A is considered a potent inhibitor of cytochrome P450, acting both as a competitive inhibitor binding to cytochrome P450 at its lipophilic site I, and as a non-competitive inhibitor (Netter, 1980). Although a different potency for some forms of P450 has been reported, SKF 525A is considered a strong inhibitor of the whole cytochrome P450 system (Murray and Reidy, 1990). In this study, SKF 525A inhibited the formation of pro-MII, MIII and 3 other skatole metabolites. These results show the involvement of cytochrome P450 in oxidative metabolism of skatole in pig liver. Particularly, the metabolic pathway leading to formation of MIII was strongly affected by both general cytochrome P450 inhibitors.

The next step of this study was to investigate the role of a P450 isozyme, CYP2E1, in skatole metabolism. This enzyme is involved in both oxidative as well as reductive metabolism. A number of substrates of CYP2E1 has been described (Koops 1992). They include both polar and non-polar compounds of different molecular structures but with a small size. This led to a suggestion that de active site of CYP2E1 acts as a molecular sieve (Guengerich and Shimada, 1991). Chlorzoxazone and DAS were used as inhibitors specific for cytochrome P450IIE1. Chlorzoxazone is a competitive inhibitor of CYP2E1 (Peter et al., 1990). Chlorzoxazone hydroxylation is also catalyzed by P450 A1 and 3A, but only at small substrate concentrations (2 μM; Jayyosi et al., 1995). At higher concentrations (0.2 mM), chlorzoxazone is metabolized almost exclusively by CYP2E1. DAS is both a competitive and via its metabolite, DASO2, a suicidal inhibitor of CYP2E1 (Brady et al., 1991). DAS has not been reported to inhibit other P450 isozymes.

Both chlorzoxazone and DAS inhibited skatole disappearance and formation of nearly all skatole metabolites in microsomal incubations, indicating CYP2E1 is involved in the synthesis of these metabolites. Whether or not other forms of cytochrome P450 are also involved in skatole metabolism can not be concluded from this study. Thornton-Manning et al. (1996) reported that eight other forms of P450 besides CYP2E1 can metabolize skatole. However, they used vaccinia-expressed human and rodent P450s and the synthesis of pro-MII was not evaluated. The strong inhibitory effect of specific CYP2E1 inhibitors compared to general P450 inhibitors on the formation of most skatole metabolites found in this study suggests that CYP2E1 is a major enzyme responsible for skatole metabolism in pigs. Moreover, the strong effect of both DAS and chlorzoxazone on pro-MII synthesis indicates that this enzyme is involved in the formation of this important metabolite of skatole.

Skatole is absorbed from the gut and metabolized in the liver and skatole which remains in the blood is quickly deposited in the fatty tissue. It is also relatively rapidly released from the fat into the blood stream and excreted in the urine in the metabolized form (Friis, 1993). We have shown that when there is a high level of CYP2E1 in liver, skatole levels in fat are low, presumably due to the rapid metabolism and clearance of skatole by CYP2E1. If CYP2E1 levels are low, skatole levels in fat can be either high or low, since the low capacity to metabolise skatole will only result in high skatole levels in fat when the amount of skatole absorbed is high. Higher levels of MII were found in the female Yorkshire pigs compared to the male pigs, while MIII and the ratio of MII/MIII differed among the females and the two groups of male pigs. However, we did not find consistent correlations between CYP2E1 levels and skatole or its metabolites in plasma. Thus, the levels of skatole metabolites in plasma, either by themselves or expressed as a ratio with skatole, cannot be used to identify those male pigs with low levels of CYP2E1 in liver. Previous suggestions that plasma metabolites of skatole could be used to find low metabolizers of skatole (Bæk et al., 1995) are not supported by our results. It might be possible to use the plasma levels of skatole or its metabolites as a marker for poor metabolizers of skatole if skatole were injected before taking plasma samples.

Levels of skatole and androstenone are higher in Wild Pig crosses than in Yorkshire pigs. The levels of androstenone and skatole are correlated in the Wild Pig crosses but not in the Yorkshire pigs. High skatole levels have been found to be related to poor growth rate and higher amount of fat (Lundström and Malmfors, 1993). Selection for faster growth and decreased backfat in Yorkshire pigs may have resulted in a decreased frequency for gene(s) causing low expression of CYP2E1. This could result in lower skatole levels in these selected pig lines compared to the wild pig crosses. It might be an advantage for wild pigs to have a high degree of ‘taint’ due to the pheromonal nature of androstenone leading to improved reproductive success. The involvement of skatole as a pheromone or for e.g. territory marking has not been demonstrated.

It appears that the differences in expression of CYP2E1 between the Yorkshire pigs and the Wild Pig crosses cannot be explained by age, but may be due to differences in sexual maturity or sex steroid hormones. While there was a significant difference in age between the two groups of animals, there was no significant correlation between age and levels of CYP2E1 within either group of animals. Levels of estrone sulfate were slightly higher in the Wild Pig crosses than in the Yorkshire pigs. Expression of CYP2E1 is induced by testosterone in mouse kidney and adrenal but not in liver (Davis and Felder, 1993). The differential effect of testosterone on expression of testosterone 15αhydroxylase (cytochrome P45015α) in mouse liver and kidney has also been reported (Squires and Negishi, 1988). In addition, the level of CYP2E1 is high in the liver of female pigs. One can therefore speculate that sex steroids cause a suppression of the amount of cytochrome P450IIE1 in the liver of some pigs.

Fat androstenone levels were correlated to levels of estrogen but not testosterone in plasma in both groups of animals. This supports a previous observation that plasma estrogen was correlated with levels of 16-androstene steroids and with boar taint determined by a trained sensory panel (Squires et al, 1991).

The present results indicate that CYP2E1 catalyzes the biotransformation of skatole in pig liver to pro-MII, an important skatole metabolite. The metabolism of skatole in the liver by this enzyme could be an important factor in regulating the concentrations of skatole in the fat of pigs. An understanding of this mechanism is important for controlling boar taint and consequently enabling the use of intact males for pork production.

### Example 2

### Conjugation Reactions in Skatole Metabolism

Materials and Methods

Chemicals

Skatole (3-methylindole), indole-3-acetonitrile, 2-naphthol, p-nitrophenol, PAPS (adenosine 3′-phosphate 5′-phosphosulfate), UDPGA (uridine 5′-diphosphoglucuronyl, trisodium salt), NADPH and NADH, Pentachlorophenol (PCP), 2,6-dichloro-4-nitrophenol (DCNP), 1-chloro-2,4-dinitrobenzene (CDNB), indole-3-carbinol and are type H-2 sulfatase from Helix pomatia vwere purchased from Sigma Chemical Co. (St. Louis, Mo., USA). 6-Hydroxy-3-methylindole (pro-MII) and 3-hydroxy-3-methyloxindole (MIII) were graciously provided by Jens Hansen Moller of the Danish Meat Research Institue, Roskilde, Denmark.

Animals

Trial I. Trial I included Yorkshire intact male pigs from a line selected for reduced backfat thickness and increased growth rate (McKay, 1990). A total of 18 pigs were selected based on the levels of skatole in backfat measured by a colorimetric procedure (Mortensen and Sorensen, 1984) . The low group included 9 males with mean skatole levels of 0.06, SD, 0.02, and range of 0.02 to 0.08 ppm. The high group consisted of 9 males with mean fat skatole levels of 0.42, SD, 0.26 and range of 0.27 to 1.07 ppm. The pigs were fed a barley, wheat and soybean meal diet containing 16% CP, 13.4 MJ DE/kg and 4% crude fiber and were slaughtered at approximately 100 kg live weight.

Trial II and III included a total of 45 F4 European Wild Pig×Swedish Yorkshire intact male pigs developed as described elsewhere (Squires and Lundström, 1997) were used. They were fed barley, oat and soybean meal diet containing 16.8% CP, 12.3 MJ DE/kg and 4.4% crude fiber and were slaughtered following stunning with carbon dioxide at approximately 108 kg live weight. The levels of skatole in backfat were measured by a colorimetric procedure (Mortensen and Sorensen, 1984). Liver samples, taken at slaughter, were frozen in liquid nitrogen and subsequently analyzed for levels of CYP2E1 by Western blotting (Squires and Lundström, 1997). Liver samples from 22 males selected for a wide range of the levels of CYP2E1 in the liver and skatole in the fat were used for enzymatic assays in Trial II, while 30 samples were used in Trial III.

Throughout the experiments the pigs were treated according to accepted standards for humane treatment of animals.

Preparation of Microsomes and Homogenates

Liver microsomes and cytosol were prepared as described by Meadus and Squires (1995). Tissues were prepared for sulfation assays in Trial 1 by homogenizing liver samples in 5 volumes of 50 mM Tris-HCL buffer, pH 7.4, 100 mM KCL, 10 mM EDTA followed by centrifugation at 10,000×g for 20 min. The supernatant was used for further study. Protein content in tissue fractions were measured with BCA Protein Assay (Pierce, Rockford, Ill., USA).

Enzyme Assays

Trial I. The rate of skatole metabolism by liver microsomes was assayed in a medium containing 50 mM Tris-HCL buffer, pH 7.4, 10 mM potassium phosphate, 0.1 mM EDTA, 20% glycerol (Buffer I), with 0.01 mM skatole and 1 mg/mL microsomal protein in a total volume of 100:1. The incubations were started, after 3 min preincubation at 37° C., by addition of 1 mM NADPH and 1 mM NADH. The reaction was terminated after 10 min by addition of 100 μl of ice cold methanol. The mixture was vortexed and centrifuged for 5 min at 2000×g to precipitate protein. Supernatant (100 μl) was analyzed by HPLC and the rate of skatole metabolism was evaluated by monitoring the changes in skatole concentration.

The activities of UDP-glucuronyltransferase were assayed in 50 mM Tris-HCL buffer, pH 7.4, 10 mM MgCl2, 0.1 mM EDTA (Buffer II). The assays with p-nitrophenol contained 0.06 mM p-nitrophenol and 1 mg/mL microsomal protein, in a total volume of 1 mL; the assays with 2-naphthol contained 0.02 mM 2-naphthol and 0.1 mg/mL microsomal protein in a total volume of 100:1. Both assays were started by addition of 0.5 mM UDPGA anti were performed at 37° C. for 5 min. The activity of sulfotransferase was assayed using 2-naphthol as a substrate. The incubation mixture contained Buffer II, from 1.9 to 3.1 mg/ml protein, 0.01 mM 2-naphthol and 1 mM PAPS, in a total volume of 100 μl. These incubations were performed at 37° C. for 60 min. The concentration of p-nitrophenol was determined by measuring the absorbance of the reaction mixture at 400 nm immediately after incubation. The assays with 2-naphthol were terminated by addition of 100:1 of ice-cold methanol, centrifuged and the concentration of 2-naphthol in the supernatant was measured by HPLC as described for the assays of skatole metabolism in microsomes.

Trial II. For evaluation of skatole metabolite production (Trial II-a, n=22), 1 mg/ml of liver microsomes were incubated in Buffer I, 1 mM NADPH, 1 mM NADH, and 0.01 mM skatole in a total volume of 2 ml for 30 min at 37° C. as previously described (Babol et al., 1997). To determine which microsomal skatole metabolites are conjugated with sulfate (Trial II-b, n=3), the reaction mixture was first incubated for 30 min as in Trial II-a. PAPS was then added to 1 mM final concentration and the incubation continued for 30, 60 and 90 min. Control incubations were performed for the same period of time with no PAPS added. Assays to evaluate the conjugation of skatole metabolites with glucuronic acid (Trial II-c, n=3) were conducted in a similar way by adding 1 mM of UDP-GA after 30 min of initial incubation and continuing the incubation for a further 7.5, 15 and 30 min. For conjugation assays in Trial II-b and II-c liver microsomes from pig; with sufficient levels of production of skatole metabolites were used. The mean levels of skatole in the fat and CYP2E1 In liver in those samples were, in Trial II-b, 0.11 (SD, 0.03 and 62.4 (SD, 41.9), respectively, and, in Trial II-c, 10 (SD, 0.04) and 101.1 (SD, 30.2), respectively.

The rate of pro-MII glucuronidation (Trial II-d, n=22) was assayed in Buffer II containing 0.1 mg ml microsomal protein, 0.5 mM UDPGA and approximately 0.5 μg/ml of pro-MII in a total volume of 2 ml. The assays were incubated at 37° C. for 5 min. The rate of pro-MIII sulfation by liver microsomes (Trial II-e, n=22) was evaluated in Buffer II containing 3 mg/ml of liver microsomes, 1 mM PAPS and approximately 1 μg/ml pro-MII in a total volume of 0.5 ml. These incubations were conducted at 37° C. for 30 min. The rate of conjugation was calculated from the amount of the products of the conjugation reaction that were formed. It was observed that pro-MIII was rapidly metabolized in microsomal incubations when no PAPS was added and the rate of this metabolism varied among the samples. Therefore, parallel incubations were performed with no PAPS added (Trial II-f, n=22). The rate of pro-MII disappearance in these incubation was defined as pro-MII degradation. Since pro-MII is somewhat unstable, the HPLC analysis was performed the same day that the incubations were carried out.

All assays in Trial II were terminated by adding an equal amount of 0.2 M ammonium acetate buffer, pH 5.0, and 5 μl of 0.01 mg/ml indole-3-acetonitrile was added as an internal standard. The metabolites were concentrated by solid phase extraction for HPLC analysis as described elsewhere (Babol et al., 1997). All assays were performed in duplicate.

Trial III. The assay of 5-OH-3MI sulfation in Trial III utilized 1 mg cytosolic protein incubated with 0.01 mM 5-OH-3MI and 1 mM PAPS in 0.05 M Tris/HCl pH 7.4 buffer containing 5 mM MgCl2 in a total volume of 250 μl. The incubations were carried out in a shaking water bath at 37° C. for 10 min and the reaction was stopped with 250 μL ice-cold acetonitrile. After the addition of acetonitrile, the mixture was vortexed and centrifuged at 4,000 g for 15 min. A 400 μL aliquot of the clear supernatant was diluted with 400 μL water and 100 μL of the mixture was analyzed immediately by high-performance liquid chromatography (HPLC) as described below. Conjugates were identified based on the following criteria: (1) no peaks corresponding to conjugates appeared when cytosol and/or PAPS were excluded from the incubations; (2) no peaks corresponding to conjugates appeared when boiled cytosol was used, and (3) the peaks corresponding to the conjugates dissapeared after addition of 10 μl of type H-2 sulfatase to the incubation mixture and further incubation for 3 h at 37° C. in a shaking water bath. Sulfation activity was measured as the formation of sulfoconjugate per minute per mg of cytosolic protein.

The effects of the phenolsulfotransferase inhibitors PCP and DCNP on the sulfation of 5-OH-3MI were determined by including different final concentrations of PCP or DCNP (0.001, 0.01, 0.1, 1, 10, 100 μM) in the assays. PCP was dissolved in methanol (final assay concentration 1%, v/v) and control incubations for PCP inihibition studies contained 1% methanol. DCNP was dissolved in incubation buffer. HPLC

Trial I. The analysis of skatole and 2-naphthol concentrations was performed by a modified HLPC method of Hansen-Moller (1992). The modifications included a different gradient profile: 0 min—buffer A, 100%; 3min—A, 85%; 11 min—A85%; 13 min—A, 20%; 15 min—A, 0%; 18 min—A, 0%; 18.5 min—A, 100%; 23 min—A, 100% and an addition of a guard column. The HPLC equipment included Spectra-Physics (San Jose, Calif., USA) SP8800 pump and autosampler, and SP4290 integrator, a Supelcosil RPLC-18, 25 cm×4.6 I.D. particle size 5 :m column (Supelco Canada Inc., Oakville, ON, Canada) and packed with the same material 4 cm×4.6 I.D. guard column. Both skatole and 2-naphthol were detected using Varian 2070 spectrofluorometer (Varian Canada Inc,. London, ON, Canada) with excitation wavelength set at 285 nm and emission wavelength set at 340 nm.

Trial II. HPLC procedures used in Trial II are described in detail elsewhere (Babol et al., 1997). The metabolites were separated with RP C18 column and detected by measuring UV absorbance at 250 nm and fluorescence with excitation and emission wavelengths set at 285 and 340 nm.

Trial III. 5-OH-skatole and its sulfate conjugate were separated using a previously reported binary gradient system method (Hernandez et al., 1991) with a reverse-phase Prodigy ODS, 5 μm, 250×4.6 mm column (Phenomenex, Torrance, Calif., USA). The mobile phase consisted of 0.05M ammonium acetate buffer pH 5.0, at a flow rate of 1.2 ml/min. A 25-min linear gradient from 10 to 60% acetonitrile was used, with initiation of the gradient 2 min after sample injection. 5-OH-skatole and its conjugate were determined by their fluorescence (excitation wavelength of 285 nm, emission wavelength of 350 nm). Typical retention times for 5-OH-skatole and its conjugate were 18.5 and 13.4 min, respectively.

Statistical analysis

All data were analyzed with the Statistical Analysis System (SAS Institute, 1995). The concentrations of skatole metabolites are expressed in arbitrary units of peak area derived from HPLC analysis. For the purpose of the analysis, some of the measurements were transformed (see below) to correct for their skewed distribution. However, all values given within the text are from untransformed data. The ratio of glucuronidation:sulfation activity was calculated in both trials to account for the competitive character of both reactions, and the ratio of pro-MII:MIII formation was calculated in Trial II to determine if it is related to fat skatole levels.

Trial I. Skatole levels in fat, skatole metabolism rate and the ratio of 2-naphthol glucuronidation:2-naphthol sulfation were transformed to common logarithms, and 2-naphthol sulfation to square root values. The differences between the pigs with high and low fat skatole levels and the rate of skatole metabolism and conjugation activities were analyzed with ANOVA using the GLM procedure.

Trial II. The effect of time of incubation of skatole metabolites with PAPS (Trial II-b) and UDPGA (Trial II-c) on the concentrations of the metabolites was analyzed using untransformed values with the GLM procedure. The model included the effect of time and sample. For the analysis of the results of Trials II-a, -d, -e and -f the following values were transformed to common logarithms: fat skatole levels, the peak areas of skatole metabolites, pro-MII, F-2, F-3, MIII, UV-1, the ratio of MII:MIII, pro-MII sulfation, the ratio of pro-MII glucuronidation:sulfation and pro-MII degradation. Stepwise regression analysis was used to evaluate the effect of cytochrome P450IIE1 and various skatole metabolites on the levels of skatole in fat. In addition, the pigs were divided according to P450IIE1 levels into 3 groups, low, medium and high. The differences in the metabolic activities among the groups were analyzed with the GLM procedure.

Results

Trial I

The intact male pigs with high fat skatole levels had a higher rate of skatole metabolism, higher activity of UDP-glucuronyltransferase towards 2-naphthol and higher ratio of 2-naphthol glucuronidation: 2-naphthol sulfation (P<0.05, Table 2) than did males with low levels of skatole in fat. The activity of UDP-glucuronyltransferase towards p-nitrophenol did not differ between the treatments. The activities of sulfotransferase towards 2-naphthol were decreased in the high as compared to the low fat skatole group. Skatole levels in fat were correlated with the rate of skatole metabolism, r=0.57, P=0.013 and the rate of glucuronidation of 2-naphthol r=0.52, P=0.028. The ratio of 2-naphthol glucuronidation:sulfation was more closely correlated to 2-naphthol sultation (r=0.87, P=0.001) than to glucuronidation (r=0.54, P=0.02).

Trial II

Skatole metabolites evaluated in Trials II-a, -b and -c of this experiment were described elsewhere (Babol et al., 1997). They included pro-MII (6-hydroxyskatole), MIII (3-hydroxy-3-methyloxindole), F-1 (indole-3-carbinol), UV-1 (3-hydroxy-3-methylindolenine), UV-3 (3-methyloxindole) and other metabolites F-2 and F-3 that have not been identified.

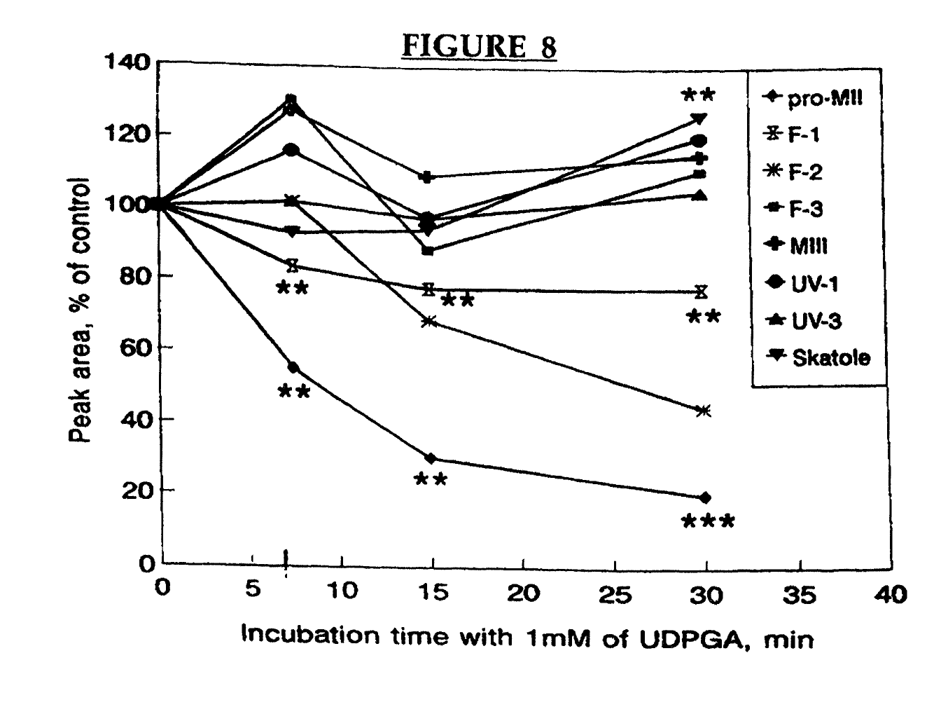

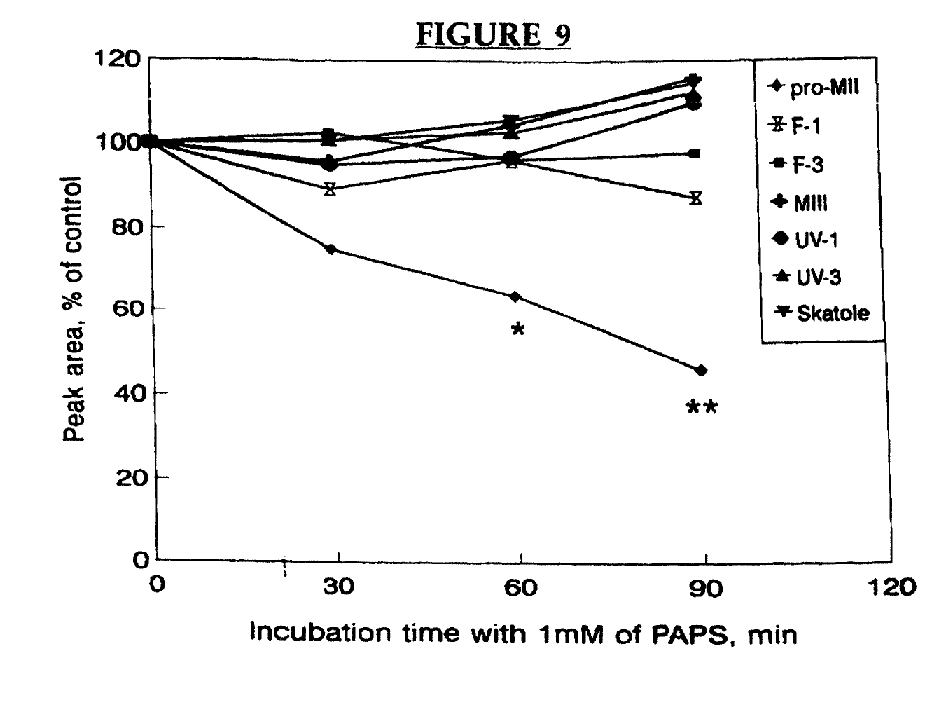

The conjugation of microsomal metabolites of skatole over time with glucuronyl (Trial II-c) and sulfate (Trial II-b) are shown in FIGS. 8 and 9. In Trial II-c, only the concentrations of pro-MII and F-1 were lower than those in control incubations (P<0.05). Mean levels of pro-MII and F-1 were both decreased after 7.5 min, and after 30 min reached 20% (SD, 25.2) and 77.8% (SD, 6.9), respectively, of those in control incubations. The mean concentrations of F-2 in 15 and 30 min incubations were numerically low (mean, 68.7%; SD, 60.2 and mean, 44.6%; SD, 39.2, respectively) but not significantly different from control (P=103). The concentration of skatole was increased in the assays with UDPGA to 126.1% (SD, 39.2) of that for control in 30 min incubation (P=0.008). In Trial II-b only the concentration of pro-MII was affected (P=0.028) and decreased to 40% (SD, 30.3) of that for control after incubating for 90 min. The mean concentrations of F-2, although numerically several times higher in sulfation assays, did not significantly differ from controls (P=0.274).

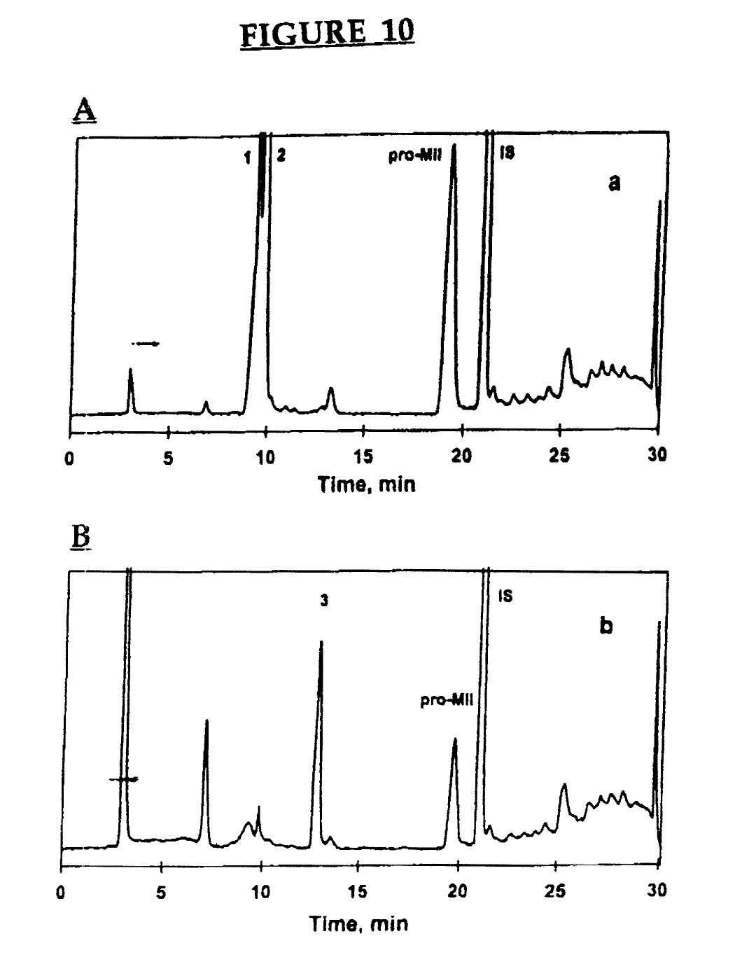

Incubation of pro-MII with liver microsomes and UDPGA (Trial II-d) resulted in production of two pro-MII metabolites (FIG. 10A). Because these were not well separated by HPLC, the sum of the peak areas of both metabolites were used to quantify the glucuronidation activity towards pro-MII. In Trial II-e, the activity of pro-MII sulfation was determined by quantification of the single product of pro-MII sulfation (FIG. 10B). The retention time from HPLC of this metabolite was the same as that of F-2 in Trial II-a, suggesting that this might be the same metabolite. In addition, the formation of this metabolite showed a strong tendency to increase in Trial II-b. The formation of this metabolite tended to be lower than in the control in Trial II-c. If F-2 is a product of pro-MII sulfation the tendency to decrease its formation observed in Trial II-b might be due to a reduced sulfation rather than to glucuronidation of pro-MII.

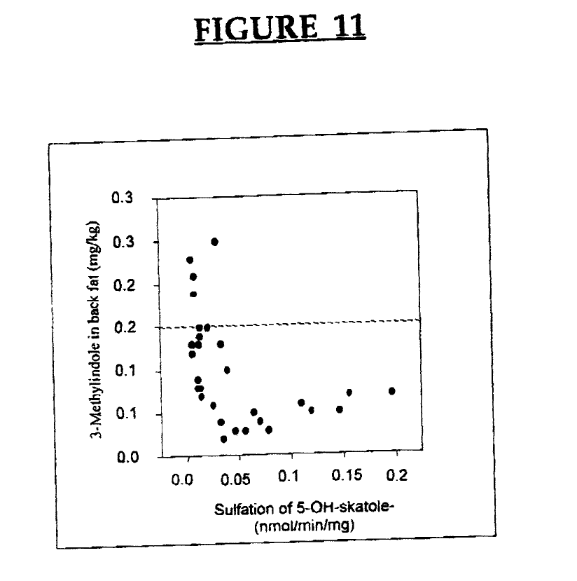

FIG. 11 shows a plot of the sulfation of 5-OH-skatole versus the concentration of skatole in fat for the 30 pigs used in the present study The PST activity, estimated as nmol of 5-sulfatoxyskatole produced per minute per mg cytosolic protein, was not significantly correlated with 3MI fat levels but pigs with high 3MI levels in fat consistently showed low sulfation activity. Conversely, pigs with high sulfation activity had low levels of 3MI in fat.

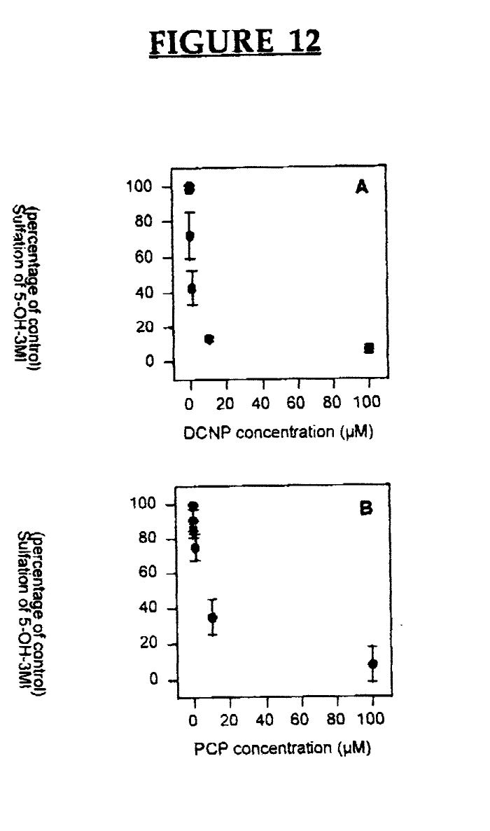

The addition of the PST inhibitors PCP or DCNP decreased the formation of 5-OH-skatole conjugate in a dose-dependent manner (FIG. 12). When no inhibitor was added, the total amount of 5-OH-skatoleI sultoconjugate produced was considered as 100%. At a concentration of 10 μM DCNP, 13.8% of the control 5-sulfatoxyskatole production was observed and at 100 μM, 7.1% of the control sulfatoxyskatole was found (FIG. 12A). When a concentration of 10 μM PCP was added to the incubations, only 35.3% of the 5-sulfatoxyskatole formed in the absence of PCP was found; when PCP concentration was increased to 100 μM, only 8% of 5-sulfatoxyskatole was produced (FIG. 12B).

The results of the ANOVA analysis of the pigs divided into low (L), medium (M) and high (H) groups according to the levels of P450IIE1 in the liver are summarized in Table 3. The L pigs compared to both M and H pigs had a lower (P<0.05) rate of formation of F-1 and sulfation of pro-MII, and higher (P<0.05) levels of skatole in fat, rate of UV-1 formation, pro-MII degradation, and the ratio of pro-MII glucuronidation:sulfation.

The levels of skatole in fat were negatively correlated with the levels of CYP2E1, metabolite F-1, sulfation of pro-MII, and positively correlated with the ratio of pro-MII glucuronidation-sulfation (Table 4). The correlation between fat skatole and liver P450IIE1 levels (r=−0.79, P<0.001) was higher in this study than that reported previously for a larger number of the same pigs (r=−0.68; Squires and Lundström, 1997) because the pigs used here were selected for a wide variation in CYP2E1 and skatole levels. The levels of CYP2E1 were correlated with the rate of pro-MII sultation and negatively correlated with the ratio of pro-MII glucuronidation:sulfation but not related to the rate of skatole metabolism or formation of skatole metabolites. Skatole oxidative metabolites were correlated with each other and with pro-MII conjugation products to various degrees. The ratio of pro-MII glucuronidation:sulfation was more closely correlated with pro-MII sulfation (r=−0.97) than with pro-MII glucuronidation (r=0.67).

Stepwise regression analysis revealed an effect on fat skatole levels of broth CYP2E1 (P=0.001) and metabolite F-1 (P=0.028). The R2 for this model was 0.71, compared to 0.62 for CYP2E1 alone. No other variable was included in the model at the P<0.05 rejection level.

Discussion

This study was conducted to investigate relationships among the levels of CYP2E1, skatole metabolism, the formation of various skatole metabolites in the liver and the levels of skatole in fat. The rate of skatole metabolism by liver microsomes in pigs in this study was lower than those reported for humans and goats (Ruangyuttckarn et at., 1991). The rate of skatole metabolism was increased in pigs with high fat skatole levels and positively correlated with fat skatole levels in Trial 1, and not related to fat skatole levels in Trial 2. This indicates that the overall rate of skatole metabolism in the liver is not related to skatole clearance and deposition in fat. The positive relation between skatole metabolism rate and fat skatole levels found in Trial 1 could be a result of a substrate stimulation of the CYP2E1 activity as it is the case for a number of other chemicals (Koop, 1992). However, injecting pigs daily for 9 days with high doses of skatole did not increase skatole metabolism rate (Babol and Squires, unpublished data). Among oxidative skatole metabolites only the formation of F-1 was correlated with fat skatole levels (r=−0.59), suggesting that the ability to produce this metabolite could be important in the metabolic clearance of skatole. When included with CYP2E1 in the multiple regression model, 71% of variation in fat skatole levels was explained by this model, leaving only 29% to other factors such as diet or environmental conditions, which are known to be important (Kjeldsen, 1993). However, the effect of CYP2E1 may be overestimated in this study since the pigs were selected for a wide variation in the levels of this enzyme. Grouping the pigs according to CYP2E1 levels in the liver showed that fat skatole levels were higher in the pigs with low (L) and similar in the pigs with medium (M) and high (H) CYP2E1 levels. F-1 formation was increased in the M and H pigs compared to L pigs, further confirming its possible role in effective skatole clearance. The higher concentration of UV-1 in liver microsomal incubations from L than from M and H pigs indicate that the formation of this metabolite is related to a slow clearance of skatole, possibly because this metabolite is not easily excreted. Formation of pro-MII, MIII and the ratio of pro-MII:MIII were not correlated to fat skatole levels. The variation in the ratio of pro-MII:MIII almost exclusively depended on the variation in the pro-MII formation, judging from the close correlation of the ratio with MII (r=0.94), compared to MIII (r=−0.50). The formation of MIII seems to be irrelevant in the rate of skatole clearance, since it was formed at a similar rate in all I, M and H pigs.

On the other hand, the results of the conjugation assays strongly suggest that the subsequent conjugation of pro-MII with sulfate or glucuronic acid are important in the metabolic clearance of skatole in pigs. Pro-MII was the only metabolite of skatole that readily formed conjugates with both sulfate and glucuronic acid. F-1 also formed conjugates but only with glucuronyl, while skatole and all the other metabolites did not react with either glucuronyl or sulfate. Sulfation and glucuronidation are major pathways of metabolism of endogenous and exogenous compounds, such as steroids, bile acids, neurotransmitters, drugs and xenobiotics (Tepnly and Burchell, 1990; Falany, 1991). Pro-MII and all the other skatole metabolites found in plasma or urine, with the exception of MIII are in the conjugated form (Bæk, 1995), indicating that conjugation reactions are necessary for clearance of skatole. In this study, distinct differences in conjugation activities in the liver were found in pigs with different skatole levels in fat. In Trial I, the activities of sulfotransferase and UDP-glucuronyltransferase were investigated using common substrates. p-Nitrophenol was used for evaluating glucuronidation and 2-naphthol was used for evaluating both glucuronidation and sulfation activities. Both p-nitrophenol and 2-naphthol are phenolic compounds predominantly used to evaluate activities of phenol sulfo- and glucuronyl transferases. Structural similarities between pro-MII and phenolic compounds were the reason for choosing these substrates for evaluating liver conjugation activities in Trial I. In Trial II, pro-MII was used for the conjugation studies. In Trial III, the skatole metabolite 5-hydroxyskatole was used.

The rates of sulfation activity towards 2-naphthol in Trial I were within the range previously reported for pigs (Smith et al., 1984). In both trials, sulfation activity was higher in pigs with low skatole levels in the fat and it was negatively correlated with fat skatole levels in Trial II. This indicates a relationship between the rapid metabolic clearance of skatole and sulfation. In particular, the sulfation of pro-MII may be important as this was the only skatole metabolite that formed a sulfoconjugate in this study. Thus, it is probably the sulfation of pro-MII that results in a rapid metabolic clearance of skatole. Synthesis of pro-MII must be important also, and a tendency for increased synthesis of pro-MII in samples with low skatole levels was observed (P=0.073).

The thermostable form of PST catalyzes the O-sulfation of simple phenolic compounds (Weinshilboum and Ottorness, 1994) and it is selectively inhibited by DCNP and PCP (Koster et al., 1982, Rein et al., 1982; Meerman et al., 1983); in contrast, the thermolabile form of PST and the hydroxysteroid sulfotransferase are resistant to inhibition by these two compounds (Weinshilboum and Ottorness, 1994). The strong dose-response inhibition of the sulfoconjugation of 5-OH-skatole by DCNP and PCP strongly suggests that TS-PST is the enzyme responsible for this reaction.

In humans, comparison of platelet PST activities with those in liver of the same subjects showed that levels of platelet TS-PST activities were significantly correlated with levels of TS-PST activity in liver (r=0.79, n=14, P<0.001); however, levels of platelet TL-PST activity were not significantly correlated with TL-PST activity in the same organ (r=0.44, n =14, P >0.10) (Weinshilboum, 1990). Since the platelet is an easily accessible tissue, this finding could be of practical significance for the selection of pigs based on high platelet sulfation activity against 5-OH-skatole, which possibly would correlate with high hepatic TS-PST activity.

Human liver contains at least two isozymes of TS-PST that differ in their thermal stabilities (Campbell et al., 1987) and two different genes for the TS-PST are now recognized in humans (hTSPST1 and hTSPST2; Weinshilboum et al., 1997). This fact indicates the possibility that a structural gene polymorphism for TS-PST may be responsible for the observation that individual subjects have either one, or both of these isoforms of TS-PST in the liver (Weinshilboum, 1990) and therefore express different sulfation activities. The same kind of polymorphism could possibly exist in the porcine species and might explain the large interindividual variation in p-nitrophenol and 5-OH-skatole sulfation activities observed in the present study.

Although sulfation appears to be the major conjugation reaction involved in skatole metabolism, glucutonidation is also involved. Pro-MII has been found in pigs not only as a sulfate but also as a glucuronyl conjugate (Bæk et al., 1995) and it was easily glucuronidated in this study. Glucuronidation and sulfation are known as competing reactions (Tamellini et al., 1991b), so whether a substrate is glucuronidated or sulfated can depend on the relative activities of both enzymes. Such a competition towards pro-MII was suggested in this study, since a trend for reduced formation of the sulfate conjugate of pro-MII was observed in the assays of skatole metabolites with UDPGA (P=0.103). UDP-glucuronyl transferases are a group of proteins that are very closely related structurally, but are functionally heterogeneous (Tephly and Burchell, 1990). This heterogeneity was observed in Trial I as p-nitrophenol glucuronidation did not differ between the treatments while 2-naphthol glucuronidation did. The rates of p-nitrophenol glucuronidation corresponded to those reported for pigs previously (Smith at al., 1984). 2-Naphthol formed a conjugate with glucuronyl faster in pigs with high than with low fat skatole levels and glucuronidation was positively correlated with fat skatole levels. A similar relationship was indicated in Trial II. The pigs with high skatole levels (L) tended to have a higher rate of glucuronidation of pro-MII than the other treatments (P=0.069). If it is the sulfation of pro-MII that is required for effective skatole clearance, formation of the glucuronyl conjugate of pro-MII may have an opposite effect to sulfation on fat skatole levels. In both trials, the ratios of glucuronidation-sulfation were higher in pigs with high skatole levels. The ratios were more closely correlated with sulfation than with glucutoridation activities indicating that it is the sulfotransferase activities that vary the most and determine whether pro-MII is sulfated or glucuronidated. Additionally, sulfation but nor glucuronidation of pro-MII was correlated with pro-MII formation, CYP2F1 and negatively correlated fat skatole levels in Trial 2. Therefore, glucuronidation of the skatole metabolite F-1 may not be important in the metabolic clearance of skatole although F-1 synthesis seems to have a positive effect Confirmation of the role of F-1 in the clearance of skatole would require further study

Pro-MII was also degraded when no cofactors were added to the microsomal incubations. The differences in pro-MII degradation were seen among pigs with different levels of CYP2E1 in liver and skatole in fat and were correlated with several microsomal skatole metabolites This suggests that pro-MU degradation might be due to the action of enzymes involved in skatole metabolism. It appears that pro-MII could be partially metabolized and partially conjugated. Both transformations could be important in the clearance of skatole, since higher rates of pro-MII degradation were found in the low skatole group of pigs.

These results indicate that both oxidation and conjugation reactions are involved in skatole metabolism and affect the levels of skatole in the fat. The expression of pig liver CYP2E1, responsible for the oxidative skatole metabolism, is probably genetically regulated, as it has been indicated in humans (Stephens et al., 1994).

Generic polymorphisms and a high degree of inheritance have been reported for both sulfotransferases (Falany, 1991; Nonneman et al., 1995) and UDP glucuronyl-transferases (Matsui and Watanabe,1982, Kroemer and Klotz 1992) in several mammalian species.

### Example 3

### Effect of Ethanol Treatment of Skatole Levels in the Plasma of Pigs

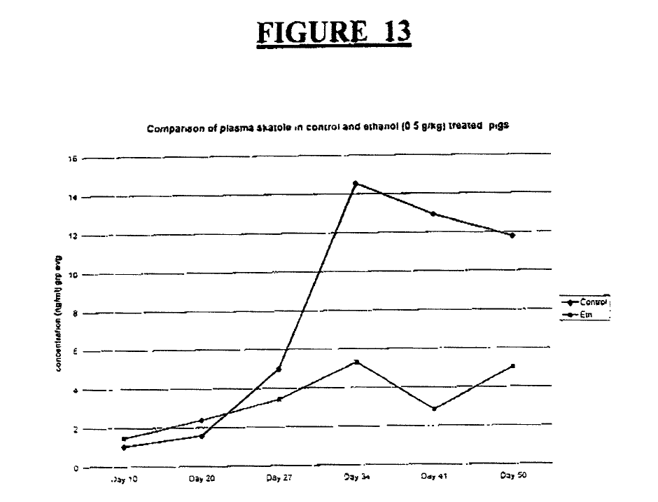

Eight uncastrated Yorkshire male pigs were utilized in the study. Four males were assigned to the control group while the remaining four were treated with 0.5g/kg BW of ethanol daily by gavage. Ethanol is a well-established inducer of CYP2E1. Treatment began at day 14 and continued until day 50. Blood samples were collected weekly and plasma skatole levels were measured by HPLC. Plasma levels in the control and treatment groups from day 10 to day 50 are plotted in a graph in FIG. 13. Skatole levels in the control group increased sharply from day 27 to day 34 and remained elevated throughout the remainder of the experiment In contrast, plasma skatole levels in the ethanol-treated group did not increase after day 27 and were significantly lower (P<0.5) than the control group.

The results of the present experiment show that the induction of CYP2E1 by ethanol treatment was effective in increasing skatole metabolism and clearanc. Therefore administration of a substance that increases the activity of the enzyme, CYP2E1, is useful for treating boar taint.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Formation of MII and MIII and the disappearance of skatole in
microsomal incubations with different cytochrome P450 inhibitors.

| | | | Metabo- | |
|---|---|---|---|---|
| | | | lite^{a} | |
| Inhibitor | Concentration, mM | pro-MII | MIII | Skatole |
| SKF 252A | .025 | 59.5* | 101.2 | 107.0 |
| | .05 | 49.7* | 73.6* | 104.6 |
| | .1 | 38.7* | 64.3* | 106.1 |
| | .2 | 38.2* | 57.1* | 115.3 |
| P-value^{b} | | .013 | .005 | .692 |
| SE^{c} | | 4.7 | 4.6 | 6.6 |
| Metyrapone | .025 | 25.8* | ND^{d} | 105.5 |
| | .05 | 14.7* | ND | 115.8* |
| | .1 | 11.6* | ND | 129.4* |
| P-value | | .001 | ND | .008 |
| SE | | 4.2 | ND | 3.9 |
| Chlorzoxazone | .025 | 82.1* | 106.5* | 102.9 |
| | .05 | 74.4* | 102.6 | 105.6 |
| | .1 | 19.6* | 62.7* | 113.2* |
| | .2 | 9.7* | 56.8* | 126.7* |
| P-value | | .001 | .001 | .007 |
| SE | | 2.8 | 1.81 | 3.82 |
| DAS | .025 | 49.6* | 80.2 | 112.3* |
| | .05 | 40.4* | 52.3* | 118.8* |
| | .1 | 35.6* | 44.2* | 132.1* |
| | .2 | 30.9* | 30.7* | 133.4* |
| P-value | | .001 | .001 | .001 |
| SE | | 3.7 | 6.1 | 1.2 |

^{a}Values are expressed as the least-squares means (n = 3) % of the peak area of control incubations.
^{b}Probability value for the differences among concentrations, including control.
^{c}Standard error of lease-squares mean.
^{d}Not determined.
*Values different from control (P < 05).

TABLE 2
Rates of skatole metabolism and activities of UDP-glucuronyltransferase
and sulfotransferase in the liver of intact males with
high and low levels of skatole in fat, Trial 1

| | High | Low | | |
|---|---|---|---|---|
| Item | (n = 9) | (n = 9) | SE^{a} | P-value |
| Level of skatole in fat, ppm^{b} | .42 | .06 | .06 | .001 |
| Rate of skatole metabolism, | .325 | .128 | .55 | .023 |
| nmol/mg protein/min^{b} | | | | |
| Rate of p-nitrophenol | 8.47 | 7.53 | .62 | .307 |
| glucuronide formation, | | | | |
| nmol/mg protein/min | | | | |
| Rate of 2-naphthol glucuronide | 29.5 | 22.1 | 1.9 | .012 |
| formation, nmol/mg protein/min | | | | |
| Rate of 2-naphthol sulfation, | .068 | .155 | .03 | .049 |
| nmol/mg protein/min^{c}. | | | | |
| Ratio of 2-naphthol glucuronide | 5062 | 211 | 3034 | .026 |
| formation:2-naphthol sulfation^{b} | | | | |

^{a}Standard error of the least-squares mean.
^{b}Statistical comparisons based on data transformed to common logarithms.
^{c}Stalistical comparisons based on data transformed to square root values.

TABLE 3
Levels of skatole in fat and the formation of skatole
metabolitesa in liver microsomes from intact male pigs with
different levels of cytochrome P450IIE1 in the liver, Trial 2
(Least-squares means ± standard errors)

| | Low | Medium | High | |
|---|---|---|---|---|
| Item | (n = 7) | (n = 8) | (n = 7) | P-value |
| P450IIE1 in | 11.7^{c} ± 5.2 | 53.0^{d} ± 4.8 | 99.5^{c}± 5.2 | .001 |
| liver^{f} | | | | |
| Skatole in fat, | .27^{c} ± .03 | .13^{d} ± .03 | .09^{d} ± .03 | .001 |
| ppm^{f} | | | | |
| pro-MII^{f} | 9.9 ± 5.1 | 28.7 ± 4.7 | 21.4 ± 5.1 | .073 |
| F-1 | 126^{d} ± 46 | 352^{c} ± 43 | 278^{c} ± 46 | .007 |
| F-2^{f} | 126 ± 20.4 | 35.6 ± 19.1 | 36.2 ± 20.4 | .327 |
| F-3^{f} | 144 ± 24 | 70 ± 23 | 103 ± 24 | .087 |
| MIII^{f} | 20.2 ± 2.7 | 20.5 ± 2.5 | 17.7 ± 2.7 | .618 |
| UV-1^{f} | 30.6^{c} ± 3.2 | 16.8^{d} ± 3.0 | 18.0^{d} ± 3.2 | .023 |
| UV-5 | 43.4 ± 4.8 | 27.9 ± 4.4 | 34.1 ± 4.8 | .084 |
| Ratio pro- | .48 ± .38 | 1.58 ± .35 | 1.55 ± .38 | .141 |
| MII:MIII^{f} | | | | |
| Skatole | .198 ± .024 | 156 ± .22 | .147 ± .024 | .311 |
| metabolism, | | | | |
| nmol/mg | | | | |
| protein/min | | | | |
| pro-MII | 201^{d} ± 375 | 936^{c} ± 351 | 1172^{c} ± 375 | .004 |
| sulfation^{f} | | | | |
| pro-MII | 1347 ± 1396 | 873 ± 1305 | 1072 ± 1396 | .069 |
| glucuronidation | | | | |
| Ratio pro-MII | 19.5^{c} ± 2.9 | 1.9^{d} ± 2.7 | 3.1^{d} ± 2.9 | .003 |
| glucsulf^{f} | | | | |
| pro-MII | 285^{c} ± 29.6 | 127^{d} ± 27.7 | 117^{d} ± 29.6 | .013 |
| degradation^{f} | | | | |

^{a}Concentrations of skatole metabolites expressed in arbitrary units of peak area derived from HPLC analysis.
^{b}Concentrations expressed in arbitrary units derived from Western blotting analysis (Squires and Lundstrom, 1997).
^{cde}Means with different superscripts within a raw differ (P < .05).
^{f}Statistical comparisons based on data transformed to common logarithms.

TABLE 4
Correlations between the various substances in male pigs. Wild Pig crosses below and
Swedish Yorkshire pigs above the diagonal

| | Skatole in | Androst- | | Testost- | Estrone | | | Ratio |
|---|---|---|---|---|---|---|---|---|
| | fat | erone | CYP2E1 | erone | sulphate | MII | MIII | MII/MIII |
| Skatole in fat, | | 16 NS | −.04 NS | −.35 NS | −.02 NS | .32 NS | 29 NS | −17 NS |
| ppm¹ | | n = 24 | n = 13 | n = 30 | n = 30 | n = 30 | n = 30 | n = 30 |
| Androsterone in | 45** | | .29 NS | .35 NS | .60** | −21 NS | −.26 NS | 20 NS |
| fat, ppm¹ | n = 45 | | n = 11 | n = 24 | n = 24 | n = 24 | n = 24 | n = 24 |
| P450IIE1 in liver, | −68*** | −.56*** | | .33 NS | .07 NS | −41 NS | −16 NS | −.01 NS |
| arbitrary units | n = 32 | n = 32 | | n = 13 | n = 13 | n = 13 | n = 13 | n = 13 |
| Testosterone | .27 NS | .29 NS | −46 NS | | .35 NS | −34 NS | −.57*** | 47** |
| in plasma, nmol/L | n = 21 | n = 21 | n = 16 | | n = 30 | n = 30 | n = 30 | n = 30 |
| Estrone sulphate | 53* | 69*** | −.57* | .40 NS | | −.29 NS | −.48** | 40* |
| in plasma, nmol/L | n = 21 | n = 21 | n = 16 | n = 21 | | n = 30 | n = 30 | n = 30 |
| MII in plasma | −.11 NS | .01 NS | .10 NS | −40* | −.16 NS | | 49** | −.04 NS |
| μg/ml¹ | n = 21 | n = 21 | n = 16 | n = 21 | n = 21 | | n = 30 | n = 30 |
| MIII in plasma, | 43* | .26 NS | −38 NS | 57** | 26 NS | −01 NS | | −89*** |
| μg/ml¹ | n = 21 | n = 21 | n = 16 | n = 21 | n = 21 | n = 21 | | n = 30 |
| Ratio MII/MIII, | −.42 NS | −.20 NS | 36 NS | −69*** | −31 NS | 59** | −81*** | |
| μg/ml¹ | n = 21 | n = 21 | n = 16 | n = 21 | n = 21 | n = 21 | n = 21 | |

*P < .05, **P < .01; P < .001.
¹Correlations based on data transformed to common logarithms.

### FULL CITATIONS FOR REFERENCES REFERRED TO IN THE SPECIFICATION

Andersson, L., C. S. Haley, H. Ellegren, S. A. Knott, M. Johanson, K. Anderson, L. Andersson-Ekund, I. Edfors-Lilja, M. Fredholm, I. Hanson, J. HÂansson, and K. Lundström. 1994. Genetic mapping of quantitative trait loci for growth and fatness in pigs. Science 263:1771-1774.

Babel, J., E. J. Squires and K. Lundström. 1998. Hepatic metabolism of skatole in pigs by cytochrome P450IIE1. J. Anim. Sci. 76:822-828

Bæk, [symbol] ., J. Hansen-Møller, C. Friis, and S. H. Hansen. 1995. Identification and quantification of selected metabolites of skatole—possibilities for metabolic profiling of pigs. Proc. EAAP Working Group: Production and Utilization of Meat from Intact Male Pigs, Milton Keynes, INRA and MLC.

Bonneau, M. 1997. Proc. EAAP Working Group on the Production and Utilization of Meat from Entire Male Pigs, Stockholm.

Brady J. F., H. Ishizaki, J. M. Fukuto, M. C. Lin., A. Fadel, J. M. Gapac, and C. S Yang. 1991. Inhibition of cytochrome P450 2E1 by diallyl sulfide and its metabolites. Chem. Res. Toxicol. 4:642-647.

Campbell, N. R. C., Van Loon, J. A. and Weinshilboum, R. M. 1987. Human liver phenol sulfotransferase: assay conditions, biochemical properties and partial purification of isozymes of the thermostable form. Biochemical Pharmacology, 36:1435-1446.

Claus, R., U. Weiler, and A. Herzog. 1994. Physiological aspects of androstenone and skatole formation in the boar—a review with experimental data. Meat Sci. 38:289-305.

Davis, J. F. and M. R. Felder. 1993. Mouse ethanol-inducible cytochrome P-450 (P450IIE1) Characterization of cDNA clones and testosterone induction in kidney tissue. J. Biol. Chem. 268:16584.

Dutton, G. J. 1966. The biosynthsis of glucurouides. In: G. J. Dutton (ad.) Glucuronic Acid, Ch. 3. pp 186-299 Academic Press, New York.

Falany, C. N. 1991. Molecular enzymology of human liver cytosolic sulfotransferases. Trends Pharmacol. Sci. 12:255-259.

Friis, C. 1993. Distribution, metabolic fate and elimination of skatole in the pig. In: M. Bonneau (Ed.) Measurement and prevention of boar taint in intact male pigs. p 113-115. INRA Edition, Paris.

Guengerich, F. P., and T. Shimada. 1991. Oxidation of toxic and carcinogenic chemicals by human cytochrome P-450 enzymes. Chem. Res. Toxicol. 4:391-407.

Halpert, J. R., Guengerich, F. R., Bend J. R. and Correia, M. A. 1994. Selective inhibitors of cytochromes P450. Toxicology and Applied Pharmacology, 125:163-175.

Hansen, L. L., A. E. Larsen, B. B. Jensen, and J. Hansen-Møller. 1995. Short time effect of an antibiotic feed additive and heavy fouling with feces plus urine on boar taint in male pigs with high and low basic skatole levels in blood. Proc. EAAP Working Group. Production and Utilization of Meat from Intact Male Pigs, Milton Keynes, INRA and MLC.

Hernandez, S. J., Powers, S. P., and Weinshilboum, R. M. 1991. Human liver arylamine N-sulfotransterase activity. Thermostable phenol sulfotransferase catalyzed the N-sulfation of 2-naphthylamine. Drug Metabolism and Disposition, 19:1071-1079.

Jayyosi, Z., D. Knoble, M. Muc, J. Erick, P. E. Thomas, and M. Kelley. 1995. Cytochrome PS450 2E1 is not the sole catalyst of chlorzoxazone hydroxylation in rat liver microsomes. J. Pharmacol Exp. Ther. 273:1156-1161.

Jensen, M. T. and B. B. Jensen. 1995. Microbial production of skatole in the hind gut of pigs fed different diets and its relation to skatole deposition in backfat. Proc. EAAP Working Group Production and Utilisation of Meat from Entire Male Pigs Milton Keynes, INRA and MLC.

Joshi, H. S., and J. I. Reaside. 1973 Synergistic effect of testosterone and oestrogens on accessory sex glands and sexual behaviour of the boar. J. Reprod. Fertil. 33:411-423.

Kjeldsen, N. 1993. Practical experience with production and slaughter of intact male pigs. In: M. Bonneau (Ed.) Measurement and prevention of boar taint in intact male pigs p 137-144. INRA Edition, Paris.

Knecht, K. T., R. C. Thurman, and R. P. Mason. 1993. Role of superoxide and trace transition in the production of (-hydroxyethyl radical from ethanol by microsomes from alcohol dehydrogenase-deficient deer mice. Arch. Biochem. Biophys. 303:339-348.

Koop, D. R. 1992. Oxidative and reductive metabolism by cytochrome P450 2E1. FASEB J. 6:724-730.

Koster, H., Halsema, I., Scholtens, E., Meerman, J. H. N., Pang, K. S., and Mulder, G. J. 1982. Selective inhibition of sulfate conjugation in the rat. Pharmacokinetics and characterization of the inhibitory effect of 2,6-dichloro-4-nitrophenol. Biochemical Pharmacology, 31:1919-1924.

Kroemer, H. K., and U. Klotz. 1992. Glucuronidation of drugs A re-evaluation of the pharmacological significance of the conjugates and modulating factors. Clin. Pharmacokinet. 23:292-310.

Lundström, K., and B. Malmfors. 1993. Skatole levels as affected by inheritance and season. Proc. 39th International Congress of Meat Science and Technology, Calgary, paper S2P13.WP.

Lundström, K., B. Malmfors, S. Stern, L. Rydhmer, L. Eliasson-Selling, A. B. Mortensen, and H. P. Mortensen. 1994. Skatole levels in pig5 selected for high lean tissue growth rate on different protein levels. Livest. Prod. Sci. 38:125-132.

Matsui, M., and H. Homma. 1994. Biochemistry and molecular biology of drug metabolizing sulfotransferase. Int. J. Biochem. 26:1237-1247.

Matsui, M., and H. K. Watanabe. 1982. Clasification of genetic expression of Wistar rats with high and low hepatic microsomal UDP-glucuronosyltransferase activity towards androsterone. Biochem. J. 202:171-174.

McKay, R. M. 1990. Responses to index selection for reduced backfat thickness and increased growth rate in swine. Can. J. Anim. Sci. 70:973-977.

Meadus, W. J. and J. E. Squires. 1995. Effect of phospholipids and organic solvents on the formation of 5,16-androstadien-3b-ol from pregnenolone in adrenal and testicular microsomes. Steroids 60:395-400.

Meerman, J. H. N., Sterenborg, H. M. J., and Mulder, G. J. 1983. Use of pentachlorophenol as long-term inhibitor of sulfation of phenols and hydroxamic acids in the rat in vivo. Biochemical Pharmacology, 32:1587-1593

Mortensen, A. B. and S. E. Sorensen. 1984. Relationship between boar taint and skatole determination with a new analysis method. Proc. 30th Eur. Meet. Meat Res. Workers, Bristol, Paper 8-11, p 394-396.

Murray, M., and G. F. Reidy. 1990. Selectivity in the inhibition of mammalian cytochromes P-450 by chemical agents. Pharmacol. Rev. 42:85-101.

Netter, K. J. 1980. Inhibition of oxidative drug metabolism in microsomes. Pharmac. Ther. 10:515-535.

Nonneman, D. H., Shibuya, H., and G. S. Johnson. 1995 A Ms1IPCXR/RFLP in the bovine phenol sulfotransferase gene. Anim. Genetics 25:436.

Pacifici, G. M., Tamellini, A., Castiglioni. M., D'Alessandro, C., Ducci, A., and L. Guliani. 1994. Interindividual variability of the human hepatic sulphotransferses. Chem-Biol Interact. 92:219-231.

Raucy, J. L., C. Curley, and S. P. Carpenter. 1995. Use of lymphocytes for assessing ethanol-mediated alteration in the expression of hepatic cyrochrome P452E1. Alcohol. Clin. Exp. Res. 19:1369-1375.

Rein, C., Glover, V., and Sandler, M. 1982. Multiple forms of phenolsulfotransferase in human tissues. Selective inhibition by dichloronitrophenol. Biochemical Pharmacology, 31:1893-1897.

Roos, P. H., B. Golub-Ciosk, P. Kallweit, D. Kuaczinski, and W. G. Hanstein. 1993. Formation of ligand and metabolite complexes as a means for selective quantitation of cytochrome P450 isozymes. Biochem. Pharmacol. 45:2239-2250.

Runge-Morris, M. A., 1997. Regulation of expression of the rodent cytosolic sulfotransferases. FASEB J.11:109-117.

Ryan, D. E., S. Lida, A. W. Wood, P. E. Thomas, C. S Lieber, and W. Levin. 1984 Characterization of three highly purified cytochromes P450 from hepatic microsomes of adult male rats. J. Biol. Chem. 259:1239-1250.

SAS Institute. 1995. SAS system for Windows, release 6.11, SAS Institute Inc, Cary, NC.

Skiles, G. L., J. D. Adams Jr., and G. S. Yost. 1989. Isolation and identification of 3-hydroxy-3-methyloxindole, the major murine metabolite of 3-methylindole. Chem. Res. Toxicol. 2:254-259.

Smith, D. J., G. L. Skiles, M. L. Appleton, J. R. Carlson, and G. S. Yost. 1993. Identification of goat and mouse metabolites of the pneumotoxin, 3-methylindole. Xenobiotica 3:1025-1044.

Smith, D. J., G. L. Skiles, M. L. Appleton, J. R. Carlson, and G. S. Yost. 1993. Identification of goat and mouse metabolites of the pneumotoxin, 3-methylindole. Xenobiotica 23:1025-1044.

Squires, E. J. and M. Negishi. 1988. Recriprocal regulation of sex-dependent expression of testosterone 15α-hydroxylase (P-45015α) in liver and kidney of male mice by androgen. J. Biol. Chem. 263:4166.

Squires, E. I., and K. Lundström. 1997. Relationship between cytochrome P450IIE1 in liver and levels of skatole and its metabolites in intact male pigs. J. Anim. Sci. 75:2506-2511.

Squires, E. I., E. A. Gullett, K. R. S. Fisher and G. D. Partolw. 1991. Comparison of androst-16-ene steroid levels determined by a colorimetric assay with boar taint estimated by a trained sensory panel. J. Anim. Sci. 69:1092.

Stephens, E. A, J. A. Taylor, N. Kaplan, C. H Young, L. L. Hsieh, G. W. Lucier, and D. A. Bell. 1994. Ethnic variation in the CYP2E1 gene: polymorphism analysis of 695 African-Americans, European-Americans and Taiwanese. Pharmacogenetics 4:185-192.

Tarnellini, A., L. Giuliani, and G. M. Pacifici. 1991a. Interindividual variability in the glucuronidation and sulphation of ethinyloestradiol in human livers. Br. J. Clin. Pharmacol. 31:661-664.

Tarnellini, A., M. Franchi, L. Guliani, and G. M. Pacifci. 1991b. Human liver sulphotransferase and LTDP-glucuronosyltransferase; structure-activity relationship for phenolic substrates. Xenobiotica 21:171-177.

Tephly, T. R, and B. Burchell 1990. UDP-glucaronosyltransferases: a family of detoxifying enzymes Trends Pharmacol. Sci. 11:276-279.

Thornton-Manning, J. M. L. Appleton F. J. Gonzalez, and & S. Yost. Metabolism of 3-methylindole by vaccinia- expressed P450 enzymes: correlation of 3-methyleneindolamine formation and protein-binding 1996. J. Pharmacol. Exp Ther. 276:21-29.

Weinshiboum, R. 1990. Sulfotransferase pharmacogenetics. Pharmacology and Therapeutics, 45:93-107.

Weinshilboum, R. and Ottorness, D. 1994. Sulfotransferase enzymes. In: Kauffman, F. C. (Ed.) Handbook of Experimental Pharmacology, Vol. 112

Conjugation-Deconjugation Reactions in Drug Metabolism and Toxicity. Springer-Verlag, Berlin, pp. 45-78.

Weinshilboum, R. M., Otterness, D. M., Askoy, I. A., Wood, T. C., Her, C., and R. B Raftogianis. 1997. FASEB J. 11:3-14.

Xu, Z., E. J. Squires and M. Bray. 1994. Effect of dietary zinc deficiency on the expression of hepatic microsomal cytochrome p450 2E1 in rats. J. Nutr. Biochem. 5:308-313.

Yonjo, S., H. M. Rhee, D. H. Sohn and B. J. Song. 1996. Immunological detection of CYP2E1 in fresh rat lyinphocytes and its pretranslational induction by fasting. Biochem. Biophys. Res. Com. 227:541-546.

Yost, G. S. 1989. Mechanisms of 3-methylindole pneumotoxicity. Chem. Res. Toxicol. 2:273-279.

Zhu, B. T., L. A. Suchar, M. Gwynne, and M. T. Huang 1995. Effect of age and sex on the inducibility of hepanc microsomal 4-nitrophenol UDP-glucuronyltransferase activity in rats. Life Sci 57:19-24.

We claim:

1. A method for determining the susceptibility of a male pig to developing boar taint comprising:
(a) obtaining a sample from the pig; and
(b) detecting the levels of one or more enzymes selected from the group consisting of (i) CYP2E1; (ii) a thermostable phenol sulfotransferase and (iii) a glucuronyl transferase,
wherein high levels of CYP2E1 and/or high levels of a thermostable phenol sulfotransfease and/or low levels of a glucuronyl transferal indicates that the pig has a reduced susceptibility to developing boar taint.

2. A method according to claim 1 wherein the sample is selected from the group consisting of a liver sample and blood.

3. A method according to claim 2 wherein levels of CYP2E1 and/or thermostable phenol sulfotransfease that are the same or higher than in a female control pig indicate that the pig has a reduced susceptibility to developing boar taint.

4. A method according to claim 2 wherein levels of CYP2E1 and/or thermostable phenol sulfotransfease that are higher than the levels of CYP2E1 and/or thermostable phenol sulfotransfease in a male control pig with boar taint indicate that the pig has a reduced susceptibility to developing boar taint.

5. A method according to claim 2 wherein levels of CYP2E1 and/or thermostable phenol sulfotransfease that are higher than the average levels of CYP2E1 and/or thermostable phenol sulfotransfease in a group of male control pigs with boar taint indicate that the pig has a reduced susceptibility to developing boar taint.

6. A method according to claim 4 wherein levels of CYP2E1 that are two to three times higher than the average levels of CYP2E1 in a group of male control pigs with boar taint and/or levels of thermostable phenol sulfotransfease that are three to four times higher than the average levels of thermostable phenol sulfotransfease in a group of male control pigs with boar taint indicate that the pig has a reduced susceptibility to developing boar taint.

7. A method for reducing or preventing boar taint comprising enhancing the metabolism of skatole in a pig.

8. A method according to claim 7 comprising enhancing the activity of the CYP2E1 enzyme in a pig.

9. A method according to claim 8 wherein the activity of the CYP2E1 enzyme is enhanced by administering
(a) a substance that increases the activity of the CYP2E1 enzyme; or
(b) a substance that induces or increases the expression of the CYP2E1 gene.

10. A method according to claim 8 wherein a nucleic acid sequence encoding a CYP2E1 enzyme is introduced into a pig.

11. A method according to claim 7 comprising enhancing the activity of the sulfotransferase enzyme in a pig.

12. A method according to claim 11 wherein the activity of the sulfotransferase enzyme is enhanced by administering
(a) a substance that increases the activity of the sulfotransferase enzyme; or
(b) a substance that induces or increases the expression of the sulfotransferase gene.

13. A method according to claim 11 wherein a nucleic acid sequence encoding a sulfotransferase enzyme is introduced into a pig.

14. A method according to claim 7 comprising inhibiting the activity of the glucuronyl transferase enzyme in a pig.

15. A method according to claim 14 wherein the activity of the glucuronyl transferase enzyme is decreased by administering
(a) a substance that decreases the activity of the glucuronyl transferase enzyme; or
(b) a substance that reduces or decreases the expression of the glucuronyl transferase gene.

16. A method according to claim 14 wherein an antisense nucleic acid sequence that is complementary to a nucleic acid sequence encoding a glucuronyl transferase enzyme is introduced into a pig.

17. A method of screening for a substance that enhances the activity of CYP2E1 comprising assaying for a substance which selectively (i) enhances CYP2E1 activity, or (ii) enhances transcription and/or translation of the gene encoding CYP2E1.

18. A method according to claim 17 comprising the steps of:
(a) reacting a substrate of CYP2E1 and CYP2E1, in the presence of a test substance, under conditions such that CYP2E1 is capable of converting the substrate into a reaction product;
(b) assaying for reaction product, unreacted substrate or unreacted CYP2E1; and(c) comparing to controls to determine if the test substance selectively enhances CYP2E1 activity and thereby is capable of enhancing skatole metabolism in a pig.

19. A method according to claim 17 comprising the steps of:
(a) culturing a host cell comprising a nucleic acid molecule containing a nuclei acid sequence encoding CYP2E1 and the necessary elements for the transcription or translation of the nucleic acid sequence, and optionally a reporter gene, in the presence of a test substance; and
(b) comparing the level of expression of CYP2E1, or the expression of the protein encoded by the reporter gene with a control cell transfected with a nucleic acid molecule in the absence of the test substance.

20. A method of screening for a substance that enhances the activity of sulfotransferase comprising assaying for a substance which selectively (i) enhances sulfotransferase activity, or (ii) enhances transcription and/or translation of the gene encoding sulfotransferase.

21. A method according to claim 20 comprising the steps of:
(a) reacting a substrate of sulfotransferase and sulfotransferase, in the presence of a test substance, under conditions such that sulfotransferase is capable of converting the substrate into a reaction product;
(b) assaying for reaction product, unreacted substrate or unreacted sulfotransferase; and
(c) comparing to controls to determine if the test substance selectively enhances sulfotransferase activity and thereby is capable of enhancing skatole metabolism in a pig.

22. A method according to claim 20 comprising the steps of:
(a) culturing a host cell comprising a nucleic acid molecule containing a nuclei acid sequence encoding sulfotransferase and the necessary elements for the transcription or translation of the nucleic acid sequence, and optionally a reporter gene, in the presence of a test substance; and
(b) comparing the level of expression of sulfotransferase, or the expression of the protein encoded by the reporter gene with a control cell transfected with a nucleic acid molecule in the absence of the test substance.

23. A method of screening for a substance that inhibits the activity of glucuronyl transferase comprising assaying for a substance which selectively (i) inhibits glucuronyl transferase activity, or (ii) inhibits transcription and/or translation of the gene encoding glucuronyl transferase.

24. A method according to claim 20 comprising the steps of:
(a) reacting a substrate of glucuronyl transferase and glucuronyl transferase, in the presence of a test substance, under conditions such that glucuronyl transferase is capable of converting the substrate into a reaction product;
(b) assaying for reaction product, unreacted substrate or unreacted glucuronyl transferase; and
(c) comparing to controls to determine if the test substance selectively inhibits glucuronyl transferase activity and thereby is capable of inhibiting skatole metabolism in a pig.

25. A method according to claim 20 comprising the steps of:
(a) culturing a host cell comprising a nucleic acid molecule containing a nuclei acid sequence encoding glucuronyl transferase and the necessary elements for the transcription or translation of the nucleic acid sequence, and optionally a reporter gene, in the presence of a rest substance; and
(b) comparing the level of expression of glucuronyl transferase, or the expression of the protein encoded by the reporter gene with a control cell transfected with a nucleic acid molecule in the absence of the test substance.

26. A composition for reducing or preventing skatole metabolism comprising administering an effective amount of a substance selected from the group consisting of:
(a) a substance that increases the activity of the CYP2E1 enzyme;
(b) a substance that induces or increases the expression of the CYP2E1 gene;
(c) a substance that increases the activity of the sulfotransferase enzyme;
(d) a substance that induces or increases the expression of the sulfotransferase gene;
(e) a substance that decreases the activity of the glucuronyl transferase enzyme; and
(f) a substance that reduces or decreases the expression of the glucuronyl transferase gene.
